(19) United States
(12) Patent Application Publication
(10) Pub. No.: US 2016/0361342 A1
(43) Pub. Date: Dec. 15, 2016

(54) USE OF ALGINATE OLIGOMERS IN THE TREATMENT OF CYSTIC FIBROSIS AND OTHER CONDITIONS ASSOCIATED WITH DEFECTIVE CFTR ION CHANNEL FUNCTION

(71) Applicant: ALGIPHARAMA AS, Sandvika (NO)

(72) Inventors: Gunnar C. HANSSON, Gothenburg (SE); Anna ERMUND, Västra Frölunda (SE); Astrid Hilde MYRSET, Oslo (NO); Arne DESSEN, Røyken (NO); Edvar J ONSØYEN, Drammen (NO); Philip D. RYE, Eiksmarka (NO)

(21) Appl. No.: 15/121,671

(22) PCT Filed: Feb. 27, 2015
(86) PCT No.: PCT/EP2015/054207

§ 371 Date: Aug. 25, 2016

(30) Foreign Application Priority Data
Feb. 28, 2014 (GB) 1403630.5
Feb. 9, 2015 (GB) 1502110.8

(51) Int. Cl. A61K 31/715; A61K 45/06

(52) U.S. Cl. CPC A61K 31/715; A61K 45/06

(57) ABSTRACT

A method is for the treatment of a condition in a human patient arising from or associated with a defective cystic fibrosis transmembrane conductance regulator (CFTR) ion channel and/or abnormal mucus which is attached to underlying epithelium. The method includes administering an alginate oligomer, in which at least 30% of the monomer residues of the alginate oligomer are G residues, to the patient in an amount sufficient to achieve a local concentration of the alginate oligomer of 1 to 6% w/v at at least part of a mucosal surface with a defective CFTR ion channel and/or the abnormal mucus in the patient.

The present invention relates to, inter alia, the treatment of cystic fibrosis (CF) and complications thereof using alginate oligomers. The invention follows on from the recent determination that many of the problems of CF and the conditions associated therewith arise from the impaired unpacking and detachment of mucus from mucus secreting cells which arises as a result of the defective CFTR ion channel. According to the present invention we have now found that certain concentrations of certain alginate oligomers (e.g. 1% to 6% w/v of alginate oligomers in which at least 30% of the monomer residues are guluronate or guluronic acid residues (G residues)) cause mucus and its major component, mucin, to detach from epithelial cell layers lacking functional CFTR ion channels, such as those found in CF and other diseases, disorders or conditions characterised by CFTR dysfunction (i.e. in which CFTR dysfunction is a causal or mediating factor). As a result, the treated mucus is able to move more freely and more closely resemble mucus of a subject without CFTR dysfunction (e.g. a non-CF, or a healthy, subject). This transition of the abnormal mucus of a patient with CFTR dysfunction (e.g. a CF patient) to a more normal phenotype is proposed to result in the alleviation of many of the problems of, and arising from, CF including not only problems in the lung, but also in the gut, and other diseases, disorders and conditions which arise from, or are associated with, the defective ion channel and/or the abnormal mucus which characterises CF.

CF is an autosomal recessive genetic disease of humans arising from mutations in the cystic fibrosis transmembrane conductance regulator (CFTR) which affect the ability of this protein to transport chloride and bicarbonate ions across epithelial membranes and thereby also influence the balance of other ions such as sodium. Such mutations can result in insufficient numbers of CFTR at the epithelial cell surface and/or insufficient ion channel activity in the CFTR that are present at the epithelial cell surface. The perturbations in ionic balance caused by the effects of such mutations in CFTR manifest in stagnant mucus in all organs where mucus is formed and thickened secretions from glands in the liver and the pancreas. The presence of this stagnant mucus in the lungs, paranasal sinuses, gastrointestinal (GI) tract, pancreas, liver and female and male reproductive systems leads to a plethora of clinical conditions associated not only with poor quality of life but also morbidity and mortality. Indeed, most CF sufferers succumb to a complication directly associated with this stagnant mucus.

In the lungs of CF patients, the dense, attached and intractable mucus is insufficiently cleared by the mucociliary clearance system, and accumulates in the airways. This makes patients susceptible to chronic lung infections and inflammation (pneumonia), which causes bacteria, bacterial biofilm, and cell debris to become intermixed with the mucus and leads to increased sputum viscosity. In turn this eventually leads to permanent lung damage and remodelling and further to pulmonary hypertension, heart failure, and respiratory failure. Infection by Staphylococcus aureus, Haemophilus influenzae, Pseudomonas aeruginosa, Mycobacterium avium complex and Aspergillus fumigatus are common. Abnormal mucus higher up in the respiratory tract (e.g. in the bronchi) can also be susceptible to infection which in turn may lead to inflammation of mucosal surfaces (e.g. bronchitis). Response to antibiotics is often poor.

In the paranasal sinuses the abnormal mucus results in frequent blockages leading to facial pain, headaches and abnormal nasal drainage. The sinuses are often exacerbated by infection, to which the abnormal mucus is highly susceptible and this may lead to acute, subacute and chronic sinusitis (also known as rhinosinusitis). Overgrowth of the nasal tissue (nasal polyps) may also result as a consequence of the chronic inflammatory state induced from chronic sinus infection. These polyps can block the nasal passages and increase breathing difficulties.

In the GI tract the attached and abnormal mucus is thought to result in intestinal pain and even full obstruction. In neonatal subjects mucus can combine with meconium to plug the ileum (meconium ileus). In older patients intestinal blockage by intussusception and distal intestinal obstruction syndrome (DIOS) of the distal ileum is often seen. Bacterial overgrowth and complications associated with the stagnant mucus may also occur.

In the pancreas, thickened and attached mucus in exocrine secretions often blocks the pancreatic duct and reduces the amount of digestive enzymes and bile entering the GI tract. This causes accumulation of digestive enzymes in the pancreas which in turn reduces the ability of a patient to retrieve dietary nutrients (nutrient malabsorption) and can cause inflammation, and irreversible damage to the pancreas. Such inflammation and damage results in pancreatitis (both acute and chronic) and ultimately atrophy of the exocrine glands and fibrosis. Damage of the pancreas can also lead to loss of the islet cells, leading to cystic fibrosis-related diabetes.

In the liver thickened bile secretions and mucus lining may block the bile ducts, causing gallstones, and lead to liver damage and ultimately cirrhosis.

Fertility of healthy females is regulated in part by the properties of the mucus in the reproductive system, especially the mucus of the cervix. The vas deferens of male mammals contains mucus that can obstruct the vas deferens if that mucus is abnormal. The abnormal mucus caused by mutation in the CFTR gene has therefore been connected with both female and male infertility.

There is currently no cure for cystic fibrosis, although the life-threatening lung and liver disease can sometimes be resolved with a successful lung or liver transplant. Lung transplants in CF patients are however not always successful because lung infection can recur shortly after transplantation. This is usually a consequence of the use of immunosuppressant drugs to promote establishment of the transplant making the transplant susceptible to the infections that remain in the patient's respiratory tract above the newly transplanted lungs.

Pharmaceutical intervention in CF is therefore restricted to management of secondary symptoms and conditions and very few options are available to address the main underlying cause of those conditions: the abnormal mucus. For instance, lung complications are typically managed through antibiotic, antifungal, antiinflammatory and bronchodilator treatment regimes, the nutrient malabsorption caused by pancreatic complications can be treated with digestive enzyme supplements and cystic fibrosis-related diabetes may be treated by a combination of oral antidiabetic drugs (e.g. the sulfonylureas, biguanides and thiazolidinediones) and i.v. insulin. Liver complications are typically tackled as for other patients with liver disease, but little can be done once damage has occurred to any of these organs.

Other conditions beyond CF may also be characterised by, or associated with, defective CFTR channels, or CFTR dysfunction. In recent years it has been recognised that even in subjects who do not carry homozygous or compound heterozygous mutations in their CFTR alleles CFTR dysfunction at epithelial cell layers can occur and give rise to the abnormal mucus and endocrine secretions that are like or similar to those that characterise CF. This results in abnormal mucus clearance which in turn may lead to or at least contribute to breathing difficulties, CF-like symptoms and complications, and chronic inflammatory respiratory disorders including COPD (and its subtypes chronic bronchitis and emphysema), bronchiectasis and chronic sinusitis.

In some instances CFTR dysfunction is seen in subjects that have non-compound heterozygous mutant CFTR alleles. In such subjects the inherited dysfunction is mild and so is insufficient to manifest as overt CF, but is sufficient to result in mucus that is more dense, attached and intractable than normal, as well as secretions from glands in the liver and the pancreas that are thicker than normal. As discussed above in the context of overt CF, in the respiratory tract, such mucus is often insufficiently cleared by the mucociliary clearance system and so accumulates in the airways and may lead to further symptoms and complications. Similarly, the thickened mucus and exocrine secretions in the paranasal sinuses, gastrointestinal (GI) tract, pancreas, liver and female and male reproductive systems of these subjects may be sufficient to lead to mild forms of the plethora of clinical conditions associated with overt CF.

Accordingly, diseases and disorders (or more generally a “condition”) associated with a defective CFTR ion channel may include not only CF, but also other conditions involving respiratory dysfunction (more generally other respiratory disorders), and in particularly disorders involving pulmonary obstruction, including particularly asthma. Indeed, asthma has been associated with CFTR gene mutation and dysfunction.

In other instances it has been shown that CFTR dysfunction may be acquired. It is now known that the chronic inhalation of particulate irritants, e.g. smoke particles (tobacco, wood etc.), pollution, dust (asbestos, cotton, coal, stone, animal droppings etc.) and spores, can result in reduced CFTR ion channel activity at epithelial cell surfaces carrying the receptor. It will be seen that in subjects whose CFTR display mild dysfunction because of an inherited defect, these deleterious effects of environmental factors on CFTR may be more pronounced clinically. This acquired dysfunction and the effects on mucus are thought to contribute to the progression of chronic inflammatory disorders, e.g. COPD, CB, emphysema, bronchiectasis and chronic sinusitis in these subjects.

CFTR ion channel function may be reduced by inhibiting the passage of ions through the channels/pores present on the epithelial cell surface (e.g. by decreasing gating duration or probability or by interfering with ion transit when the channel is open—channel conductance) or by reducing the number of CFTR on the cell surface (e.g. by reducing expression of CFTR, interfering with the transport of functional CFTR to the cell surface and/or by upregulating the process of internalisation and turnover of CFTR from the cell surface). It has also been recognised that similar effects on CFTR processing can be seen during chronic airway inflammation, e.g. as seen in COPD (and its subtypes chronic bronchitis and emphysema), bronchiectasis and chronic sinusitis.

In all of these contexts reduced CFTR activity in the respiratory tract may result in the dense, attached and intractable mucus characteristic of CF which is insufficiently cleared by the mucociliary clearance system and which accumulates in the airways. This makes patients with acquired CFTR dysfunction susceptible to the respiratory symptoms and complications experienced by CF patients, including those shared with COPD, CB, emphysema, asthma and chronic sinusitis.

A few approaches have been developed to address the abnormalities of CF mucus, principally its elevated viscosity. These include dornase alfa (a DNase enzyme), hypertonic saline, mannitol, acetylcysteine, dextran and denufosol (an agonist of the P2Y2 subtype of purinergic receptors, an alternative chloride channel in the lung). However, these treatments only show limited efficacy and are limited to the lung. Alginate oligomers have also been shown to be capable of reducing the viscosity of sputum from COPD patients and cervical mucus (WO 2007/039754, WO 2007/039760; WO2008/125828). The use of alginate oligomers to treat CF, female infertility and hyperviscous mucus in the gut has been proposed on this basis.

In addition to pharmaceutical interventions, CF patients will typically undergo physiotherapy to the chest and/or abdomen designed to alleviate the lung and/or GI complications of CF, particularly in relation to assisting the clearing of the lungs and/or breathing. Such physiotherapy techniques may include one or more of active cycle of breathing techniques (ACBT), postural drainage, manual percussion and vibration, autogenic drainage (AD), high frequency chest wall oscillation (HFCWO), positive expiratory pressure (PEP), and oscillating positive expiratory pressure devices (Oscillating PEP).

There is therefore a continuing need for pharmaceutical interventions in this area, and in a particular interventions that can correct the abnormalities in the mucus of CF patients that give rise to the various complications outlined above, especially those associated with the lung, the GI tract, the pancreas, the liver and the reproductive system.

Likewise, there is therefore a continuing need for pharmaceutical interventions that can correct the abnormalities in the mucus of patients with CFTR dysfunction beyond CF, e.g. patients with abnormal mucus clearance in the respiratory tract and/or breathing difficulties resulting from chronic particulate inhalation, patients with chronic inflammatory respiratory disorders, e.g. COPD (and its subtypes chronic bronchitis and emphysema), bronchiectasis, asthma and chronic sinusitis, and/or patients with non-compound CFTR gene mutation heterozygosity and thereby treat said diseases, disorders and conditions and the various complications thereof outlined above, especially those associated with the lung, the GI tract, the pancreas, the liver and the reproductive system.

Although alginate oligomers have previously been proposed for use in reducing sputum viscosity in CF and COPD patients, we believe that the problems which arise in CF and COPD patients (and other patients with CFTR dysfunction at the epithelial cell layer of a mucosal surface) are principally due to impaired unpacking and detachment of mucus from the mucus-secreting cells of mucosal surfaces, rather than due to elevated mucus viscosity. As discussed in more detail below, we have determined a new mechanism by which alginate oligomers of particular composition (i.e. a composition in which at least 30% of the monomers are G residues) may operate to alleviate the effects of the abnormal mucus in CF and other diseases, disorders or conditions involving CFTR dysfunction and, importantly, that to achieve this beneficial new effect certain concentrations of such alginate oligomers are required.

Alginates are naturally occurring polysaccharides that have been found to have a number of uses, both clinical (e.g. in wound dressings, as drug carriers and in anti-heartburn preparations) and non-clinical (e.g. in food preparation). They are linear polymers of (1-4) linked β-D-mannuronic acid (M) and/or its C-5 epimer α-L-guluronic acid (G). The primary structure of alginates can vary greatly. The M and G residues can be organised as homopolymeric blocks of contiguous M or G residues, as blocks of alternating M and G residues and single M or G residues can be found interspacing these block structures. An alginate molecule can comprise some or all of these structures and such structures might not be uniformly distributed throughout the polymer. In the extreme, there exists a homopolymer of guluronic acid (polyguluronate) or a homopolymer of mannuronic acid (polymannuronate).

Alginates have been isolated from marine brown algae (e.g. certain species of Durvillea, Lessonia and Laminaria) and bacteria such as Pseudomonas aeruginosa and Azotobacter vinelandii. Other pseudomonads (e.g. Pseudomonas fluorescens, Pseudomonas putida, and Pseudomonas mendocina) retain the genetic capacity to produce alginates but in the wild they do not produce detectable levels of alginate. By mutation these non-producing pseudomonads can be induced to produce stably large quantities of alginate.

Alginate is synthesised as polymannuronate and G residues are formed by the action of epimerases (specifically C-5 epimerases) on the M residues in the polymer. In the case of alginates extracted from algae, the G residues are predominantly organised as G blocks because the enzymes involved in alginate biosynthesis in algae preferentially introduce the G neighbouring another G, thus converting stretches of M residues into G-blocks. Elucidation of these biosynthetic systems has allowed the production of alginates with specific primary structures (WO 94/09124, Gimmestad, M et al, Journal of Bacteriology, 2003, Vol 185(12) 3515-3523 and WO 2004/011628).

Alginates are typically isolated from natural sources as large high molecular weight polymers (e.g. an average molecular weight in the range 300,000 to 500,000 Daltons). It is known, however, that such large alginate polymers may be degraded, or broken down, e.g. by chemical or enzymatic hydrolysis to produce alginate structures of lower molecular weight. Alginates that are used industrially typically have an average molecular weight in the range of 100,000 to 300,000 Daltons (such alginates are still considered to be large polymers) although alginates of an average molecular weight of approximately 35,000 Daltons have been used as excipients in pharmaceuticals.

More recently alginate oligomers of smaller size (molecular mass) have been proposed for clinical use, most notably to reduce the viscosity of hyperviscous sputum such as occurs in sufferers of cystic fibrosis and other respiratory diseases (see WO 2007/039754 and WO 2008/125828) or to combat biofilm (WO 2009/068841) and multidrug resistant bacteria (WO 2010/13957).

Gustafsson, J. K., et al, J. Exp. Med, 2012, Vol 209(7), 1263-1272, has recently shown in a CFTR-mutant mouse model of CF that the mucus layer of the ileum is attached to the underlying epithelium, but in wild type mice it is fully detached. It is believed that this applies generally to all mucosal surfaces in the CF patient that is any mucus-secreting, mucus-carrying or to any extent mucus-coated surface, both internal or external, of the CF patient. It follows that any CFTR dysfunction at the epithelial cell layer of a mucosal surface, whether acquired or inherited, may result in a phenotype at that mucosal surface which is similar to that observed in the CFTR-mutant mouse model of CF.

Gustafsson also showed that by supplementing the medium bathing the apical side of ileum explants from mutant mice with sodium bicarbonate at levels predicted to be reached by wild type CFTR, the “attached” phenotype can be reversed and a wild type “detached” phenotype observed instead. Without wishing to be bound by theory, it is believed that bicarbonate provided by the CFTR ion channel plays a possibly essential role in the proper unfolding of mucin at secretion and this proper unfolding is required for normal mucus detachment. It is proposed that in CF and other disorders involving CFTR dysfunction the malfunctioning CFTR cannot provide sufficient bicarbonate to ensure proper unfolding and this prevents detachment.

As shown in the present Examples, it has now been found that, unexpectedly, alginate oligomers in which at least 30% of the monomer residues are G residues, when administered in an amount sufficient to achieve a local concentration of 1 to 6% w/v at the apical side of the epithelium, i.e. the lumen/mucus interface, of at least part of a mucosal surface affected by a lack or deficiency of or in functioning CFTR ion channels, in other words a mucosal surface displaying CFTR dysfunction, (e.g. a mucosal surface in a CF patient), are also able to cause or induce at least partial detachment of said mucus from the epithelium of said mucosal surface and thereby return the abnormal mucus of that surface (e.g. the abnormal mucus of a CF patient) to a more normal phenotype (i.e. the phenotype of a mucosal surface that is not displaying CFTR dysfunction e.g. a non-CF phenotype). This may be, at least in part, due to an effect on the unfolding of mucin at secretion. We propose that using such alginate oligomers to cause, or result in, this transition to a more normal phenotype (detachment) will directly address the various mucus-related complications of CF, and of other conditions arising from or associated with defective CFTR ion channel function (i.e. conditions as indicated above), as such complications arise directly or indirectly from the abnormal/adhered mucus which results from the CFTR dysfunction at the mucosal surfaces of such CF and other patients.

Specifically in the case of a patient whose lungs display CFTR dysfunction, e.g. in the lungs of a CF patient, it is proposed that the detached mucus layer will respond to the patient's mucociliary clearance systems in a manner substantially analogous to that observed in a healthy patient whose lungs do not display CFTR dysfunction (e.g. non-CF patient). It is further proposed that this detachment will alleviate stagnant mucus in the lungs of such a patient, and represent an especially effective treatment of the CF or other respiratory disorder suffered by the patient. As is also shown in the present Examples, such treatments seem not to affect the thickness of the mucus layer nor the epithelial cells. This is proposed to be advantageous in the context of a treatment of diseases, disorders or conditions involving abnormal mucus caused by CFTR dysfunction, e.g. CF, where the obstruction of the lumens of various conduits in affected tissues is already a clinical problem.

Thus, in a first aspect the invention provides a method for the treatment of a condition in a human patient arising from or associated with a defective CFTR ion channel and/or abnormal mucus which is attached to underlying epithelium, said method comprising administering an alginate oligomer, wherein at least 30% of the monomer residues of the alginate oligomer are G residues, to the patient in an amount sufficient to achieve a local concentration of the alginate oligomer of 1 to 6% w/v at at least part of a mucosal surface with a defective CFTR ion channel and/or said abnormal mucus in the patient, thereby to result in at least partial detachment of mucus from said mucosal surface.

The invention further provides an alginate oligomer for use in the treatment of a condition in a human patient arising from or associated with a defective CFTR ion channel and/or abnormal mucus which is attached to underlying epithelium, wherein at least 30% of the monomer residues of the alginate oligomer are G residues and said alginate oligomer is administered to the patient in an amount sufficient to achieve a local concentration of the alginate oligomer of 1 to 6% w/v at at least part of a mucosal surface with a defective CFTR ion channel and/or said abnormal mucus in the patient, thereby to result in at least partial detachment of mucus from said mucosal surface.

Expressed alternatively, the invention further provides the use of an alginate oligomer in the manufacture of a medicament for use in the treatment of a condition in a human patient arising from or associated with a defective CFTR ion channel and/or the abnormal mucus which is attached to underlying epithelium, wherein at least 30% of the monomer residues of the alginate oligomer are G residues and said medicament is administered to the patient in an amount sufficient to achieve a local concentration of the alginate oligomer of 1 to 6% w/v at at least part of a mucosal surface with a defective CFTR ion channel and/or said abnormal mucus in the patient, thereby to result in at least partial detachment of mucus from said mucosal surface.

Alternatively expressed, in these various aspects of the invention the alginate oligomer may be used for the treatment of a human patient with a condition arising from or associated with a defective CFTR ion channel and/or abnormal mucus which is attached to underlying epithelium. It will be understood in this respect that such abnormal mucus is the mucus which is characteristic of CF (mucus which has not detached from a mucosal surface), but which in view of the common underlying defect (loss or deficiency in CFTR function) may also be seen in other conditions, such as those discussed above. In particular, and as will be described in more detail below, the invention includes the treatment of any complication of such a condition. Accordingly, references herein to treating said condition include the treatment of one or more complications or clinical manifestations associated condition.

As used herein the term “condition” includes any disease, disorder or condition, whether arising due to a genetic defect or mutation, or in any other way, including an acquired condition, e.g. due to environmental and/or clinical exposure, as discussed above, for example.

A “defective CFTR ion channel” will be understood from the above to include any defect or deficiency in CFTR function, i.e. CFTR dysfunction. Thus “a defective CFTR ion channel” effectively means, and may alternatively be expressed as, “defective CFTR ion channel function”. The condition may thus be viewed as a condition characterised by CFTR dysfunction. As will be discussed in more detail below, this may include CFTR ion channels which are defective in the sense that they are non-functional or have reduced function, i.e. partially or fully lack CFTR ion channel activity (in other words in which CFTR ion channel activity is reduced or abrogated). Thus a lack of functional CFTR ion channels (which may underlie the condition) may include a lack of CFTR channels which are fully functional (i.e. display full or normal CFTR activity). Also included is the loss or depletion of functional CFTR ion channels, e.g. as a result of reduced or absent expression of the channel, or transport to the cell surface, or by increased internalisation or turnover or other processing leading to loss/depletion of CFTR ion channels from the cell surface. Essentially a condition associated with or arising from a defective CFTR ion channel includes any condition in which CFTR function or activity is reduced, whether due to a reduced number of CFTR ion channels or reduced activity in those that are present, or both. In particular a defective CFTR ion channel results in abnormal mucus, and more particularly mucus which has not detached from a mucosal surface.

In certain embodiments the condition may be CF, non-compound CFTR gene mutation heterozygosity, abnormal mucus clearance in the respiratory tract and/or breathing difficulties resulting from chronic particulate inhalation, and/or a chronic inflammatory respiratory disorder, e.g. COPD (and its subtypes chronic bronchitis and emphysema), bronchiectasis, asthma and/or chronic sinusitis. More generally the condition may be a respiratory disorder, e.g. an obstructive respiratory disorder. More particularly, in more specific embodiments the condition may be characterised by a chronic inflammatory state, airway remodelling and exacerbations due to respiratory tract infections.

In other embodiments the condition may be a mucus-related complication of the above-listed conditions. In a further specific embodiment the invention provides a treatment for mucus stasis and breathing difficulties in tobacco smokers and other subjects exposed to the chronic inhalation of particulate irritants, e.g. smoke particles (tobacco, wood etc.), pollution, dust (asbestos, cotton, coal, stone, animal droppings etc.) and spores.

Thus, in one specific embodiment of the first aspect the invention provides a method for the treatment of cystic fibrosis in a human patient, said method comprising administering an alginate oligomer, wherein at least 30% of the monomer residues of the alginate oligomer are G residues, to the patient in an amount sufficient to achieve a local concentration of the alginate oligomer of 1 to 6% w/v at at least part of a mucosal surface in the patient, thereby to result in at least partial detachment of mucus from said mucosal surface.

The invention further provides an alginate oligomer for use in the treatment of cystic fibrosis in a human patient, wherein at least 30% of the monomer residues of the alginate oligomer are G residues and said alginate oligomer is administered to the patient in an amount sufficient to achieve a local concentration of the alginate oligomer of 1 to 6% w/v at at least part of a mucosal surface in the patient, thereby to result in at least partial detachment of mucus from said mucosal surface.

Expressed alternatively, the invention further provides the use of an alginate oligomer in the manufacture of a medicament for use in the treatment of cystic fibrosis in a human patient, wherein at least 30% of the monomer residues of the alginate oligomer are G residues and said medicament is administered to the patient in an amount sufficient to achieve a local concentration of the alginate oligomer of 1 to 6% w/v at at least part of a mucosal surface in the patient, thereby to result in at least partial detachment of mucus from said mucosal surface.

Alternatively expressed, in these various aspects of the invention the alginate oligomer may be used for the treatment of a patient with CF. In particular, and as will be described in more detail below, the invention includes the treatment of any complication of CF, defined herein as any condition or disorder associated with or arising from the abnormal mucus which occurs in and characterises CF, and/or with a defective CFTR ion channel (in particular a defective CFTR ion channel which results in abnormal mucus, and more particularly mucus which has not detached from a mucosal surface). Accordingly, references herein to treating CF include the treatment of one or more complications of CF.

In other aspects the invention can be considered to provide methods and medical uses with the features of those described above for the treatment of CFTR dysfunction and/or the abnormal mucus which is attached to underlying epithelium in a human patient and/or the complications thereof.

A mucosal surface is defined herein as any surface of the human body, both internal or external, that secretes, has, carries or is to any extent coated with mucus. More specifically a mucosal surface is a tissue lining comprising epithelial cells, typically arranged as an epithelial cell layer (an epithelium), that secretes, has, carries or is to any extent coated with mucus. It will be recognised that the terms “mucous membrane” and “mucosa” may alternatively be used to refer to a mucosal surface. In accordance with the invention the mucosal surface targeted by the treatments of the invention will be affected by a lack of functional CFTR (i.e. a mucosal surface with a defective CFTR ion channel or, in other words, displaying CFTR dysfunction) and so will secrete, have, carry or be to any extent coated with the abnormal mucus characteristic of CF (mucus that is attached to the underlying epithelium).

Defective CFTR ion channel function at a mucosal surface (CFTR dysfunction) may be expressed in terms of reduced CFTR ion channel capacity, more specifically CFTR-mediated ion transport, as compared to normal or healthy mucosal surfaces, in particular a reduction that renders any such transport as insufficient to maintain normal or healthy mucus, in particular non-adhered mucus. Included in the term “CFTR-mediated ion transport” is the transport of ions through the CFTR itself, and also the transport of ions through secondary mechanisms, e.g. other ion channel proteins at the mucosal surface, that are driven by the ion concentration gradients maintained by the ions transported through the CFTR. Such ions include chloride, sodium and bicarbonate ions.

Defective CFTR ion channel function at a mucosal surface (CFTR dysfunction) may in turn be caused by any mechanism or combination of mechanisms that decreases the capacity of the population of CFTR at the cell surface to transport ions. This may include the inhibition of ion transport activity of the CFTR at the cell surface, e.g. because of a defect in the protein itself, because of an agent effecting a transient or permanent structural change in protein and/or an agent blocking the ion transport pore/channel. Mechanistically, inhibitory effects can be seen if the pore/channel is blocked to some extent (conductance is decreased) and/or if gating duration or probability is decreased. The capacity of the population of CFTR at the cell surface to transport ions may also be decreased if there are too few CFTR at the cell surface. This can occur if expression from the CFTR genes is insufficient. This can also occur if there is a defect in the CFTR gene, transcript or translation product that prevents the CFTR, of a portion of the population thereof, from reaching or inserting correctly into the cell surface. This can also occur if the machinery responsible for CFTR turnover is out of balance in favour of removal (internalisation) rather than replenishment. This latter mechanism may be a result of a defect in the CFTR protein or can be caused by environmental agents. It may also be the case that a subject with defective CFTR ion channel function at a mucosal surface has lower than normal numbers of CFTR at the mucosal surface and the CFTR within that population of CFTR have lower than normal ion transport activity.

Thus, in accordance with the invention, it can be seen that a lack of functional CFTR (e.g. fully functional CFTR) at a mucosal surface, by whatever means, can result in defective CFTR ion channel function at a mucosal surface (CFTR dysfunction) and thus insufficient CFTR ion channel capacity to maintain normal or healthy mucus, in particular non-adhered mucus. Consequently, patients with a mucosal surface affected by a lack of functional CFTR (i.e. a mucosal surface displaying CFTR dysfunction) may suffer from a condition arising from or associated with a defective CFTR ion channel and/or the abnormal mucus which is attached to underlying epithelium (namely the mucus which characterises CF).

In particular, we have determined that in order to achieve detachment of mucus from the epithelial cells of a mucosal surface affected by a lack of functional CFTR (i.e. a mucosal surface displaying CFTR dysfunction), certain local concentrations of certain alginate oligomers are required at the mucosal surface, namely 1 to 6% w/v of alginate oligomers in which at least 30% of the monomer residues are G residues. This means that the alginate oligomer is administered or delivered, such that the oligomer at, or reaching, the mucosal surface is at this concentration, more specifically the concentration at the mucus layer, or at the mucus coating, is at this range. Thus, for example, the local concentration at the lumen of at least part of a mucosal surface or at the mucus interface of at least part of a mucosal surface is at this level. In particular this concentration is achieved at the apical side of the epithelium, or mucosal surface.

Cystic fibrosis is a human disease characterised by mucus and/or exocrine secretions from the lung, pancreas and liver that have abnormal physical properties, typically increased viscosity and, in the case of mucus, adherence to the epithelium of the mucosal surface. These underlying factors manifest in, amongst other conditions, breathing difficulties, respiratory tract infections (chronic and acute, e.g. of the bronchi or of the lungs), respiratory tract inflammation (e.g. bronchial inflammation (termed bronchitis, if due to infection) or pulmonary inflammation/pneumonitis (termed pneumonia, if due to infection)), pulmonary hypertension, heart failure, respiratory failure, lung remodelling, sinus infection, sinusitis (acute, subacute and chronic), facial pain, headaches, abnormal nasal drainage, thickened faeces, constipation, bowel obstruction, nutrient malabsorption, pancreatic inflammation, pancreatitis, diabetes, gallstones, liver cirrhosis, and infertility. Decreased response to antibiotics, especially in the lungs, is also seen. The abnormal mucus and exocrine secretions arise from mutations in the cystic fibrosis transmembrane conductance regulator (CFTR) which affect the ability of this protein to transport chloride and bicarbonate ions across epithelial membranes and thereby regulate the balance of other ions such as sodium. Many such mutations of CFTR have been identified, some resulting in a more pronounced CF phenotype than others. A patient can therefore be considered to be suffering from CF if the patient has one or more, preferably 2, 3, 4, 5, 6 or more or all of the above mentioned conditions, abnormal mucus (e.g. mucus attached to epithelium at at least one mucosal surface), hyperviscous sputum or other secretions and/or exocrine secretions and a mutation in each of his/her CFTR genes.

Conveniently CF may be diagnosed by the “sweat test”. This is a routine test familiar to the person skilled in the art. Briefly, pilocarpine is placed on the skin and uptake induced by electric current. Sweat released at the treatment site in response to the pilocarpine is collected (e.g. absorbed onto a piece of filter paper) and is then analysed for its salt content. A person with CF will have salt concentrations that are one-and-one-half to two times greater than normal. More specifically, for infants up to and including 6 months of age, a chloride level of equal to or less than 29 mmol/L means CF is very unlikely; levels of 30-59 mmol/L mean that CF is possible; and levels greater than or equal to 60 mmol/L mean CF is likely. For people older than 6 months of age, a chloride level of equal to or less than 39 mmol/L means CF is very unlikely; levels of 40-59 mmol/L mean that CF is possible; and levels greater than or equal to 60 mmol/L mean CF is likely.

In accordance with the invention an infant patient (6 months old or younger) to which the treatment of the invention will be applied will have a sweat chloride level of greater than 25 mmol/L, preferably greater than 29 mmol/L, 35 mmol/L, 40 mmol/L, 45 mmol/L, 50 mmol/L, 55 mmol/L or 60 mmol/L and all other patients will have a sweat chloride level of greater than 35 mmol/L, preferably greater than 39 mmol/L, 45 mmol/L, 50 mmol/L, 55 mmol/L or 60 mmol/L.

As discussed above CFTR dysfunction has been recognised as being an underlying factor in conditions other than CF. Such dysfunction may be inherited through the inheritance of one mutated CFTR allele or may be acquired through, for example, chronic inhalation of particulates (in particular tobacco and wood smoke) and the chronic inflammation of the respiratory tract (e.g. in COPD and its subtypes CB and emphysema, bronchiectasis and chronic sinusitis).

Non-compound CFTR gene mutation heterozygosity is a clinical condition in which a subject has one CFTR allele that does not carry a mutation which effects the intracellular processing and/or cell surface ion channel activity of the protein expressed therefrom and one allele that does have a mutation that is detrimental to the intracellular processing and/or cell surface ion channel activity of the protein expressed therefrom. Such subjects do not display overt CF as defined above in so far as several of the various complications of CF are clearly seen at any one time, but heterozygous subjects will have, at least at times, a mild form of the abnormal mucus which characterises CF and so may present with mild forms of one or of the complications of CF without being sufficient severe as prompting a clear diagnosis of CF. Specifically subjects with CFTR heterozygosity have been observed as having recurrent “idiopathic” pancreatitis, congenital bilateral absence of the vas deferens, chronic sinusitis, and idiopathic bronchiectasis, but such patients may present with any of the CF complications described herein.

The CF sweat test can be used to identify patients with suspected non-compound CFTR gene mutation heterozygosity as such patients will fall between the “very unlikely” and “likely” ranges of sweat chloride levels. For an infant patient (6 months old or younger) this may be a sweat chloride level of greater than 25 mmol/L, preferably greater than 29 mmol/L, 35 mmol/L, 40 mmol/L, 45 mmol/L, 50 mmol/L, 55 mmol/L, but less than 60 mmol/L and all other patients will have a sweat chloride level of greater than 35 mmol/L, preferably greater than 39 mmol/L, 45 mmol/L, 50 mmol/L, 55 mmol/L, but less than 60 mmol/L. Genetic testing of suspected patients can then confirm the diagnosis.

COPD, also referred to as chronic obstructive lung disease (COLD), and chronic obstructive airway disease (COAD) is a collective term for chronic obstructive lung diseases characterised by chronic inflammation of the airways without dilation, chronically poor airflow and enhanced sputum production. It is generally accepted that the conditions of chronic bronchitis (inflammation of the mucous membranes of the bronchi) and emphysema (breakdown of the lung tissue, specifically the alveoli) are subtypes of COPD. COPD is usually diagnosed as chronically poor lung function that is not improved by administration of bronchodilators and a chronic productive cough. Imaging of the chest, e.g. with MRI and high resolution computerised tomography (HRCT) may also reveal physiologies characteristic of COPD and to rule out other respiratory conditions.

Presently COPD is not reversible and patients deteriorate over time, ultimately succumbing to respiratory failure. The enhanced sputum production observed in COPD and its similar characteristics to CF mucus mean the respiratory complications observed in CF as discussed above are common in COPD patients, in particular the complications linked to infection of the airways.

Bronchiectasis is a disease characterised by chronic enlargement and subsequent breakdown of the bronchi as a result of an inflammatory response, chronically poor lung function that may improve by administration of bronchodilators and a chronic productive cough. Diagnosis is usually based on lung function tests and imaging of the chest, e.g. with MRI and high resolution computerised tomography (HRCT) to reveal the enlarged bronchi characteristic of the disease.

Presently bronchiectasis is not reversible and patients deteriorate over time, ultimately succumbing to respiratory failure. The enhanced sputum production observed in bronchiectasis and its similar characteristics to CF mucus mean the respiratory complications observed in CF as discussed above are common in bronchiectasis patients, in particular the complication linked to infection of the airways.

Chronic sinusitis is the long term, more than three months, inflammation of the paranasal sinuses. The cause of that inflammation may be infection, allergy (usually to particulates including dust, pollution, pollen, spores and microorganisms) or an autoimmune response. The inflammation leads to increased mucus production and impaired sinus drainage and secondary bacterial infections, which further contribute to the inflammatory response. That the sinus mucus of a patient with chronic sinusitis has similar characteristics to CF mucus means the respiratory, and especially the paranasal sinus, complications observed in CF as discussed above are common in patients with chronic sinusitis. A diagnosis of chronic sinusitis is usually confirmed with nasal endoscopy.

Asthma is a chronic airway disease that manifests as acute episodes of air flow obstruction due to transient bronchoconstriction resulting from the tightening of smooth muscle surrounding the airways, predominantly the bronchioles. Such exacerbations are often triggered by exposure to external stimuli. Bronchial inflammation also leads to tissue swelling and oedema thus causing further obstruction. Underlying the overt episodes of bronchoconstriction and airway obstruction are chronic symptoms of airway thickening and remodelling due to scarring and inflammation and overdeveloped mucus glands.

There is currently no cure for asthma and treatment is limited to control of the acute symptoms. The chronic inflammatory processes and tissue remodelling of the airways associated with asthma long term, including enhanced sputum production, mean the respiratory complications observed in CF as discussed above may be seen in asthma patients, in particular the complications linked to infection of the airways.

It has also been recognised that inhalation of particulate irritants, e.g. smoke particles (tobacco, wood etc.), pollution, dust (asbestos, cotton, coal, stone, animal droppings etc.) and spores can result in defective CFTR ion channel function (CFTR dysfunction) through the inhibition of CFTR ion transport activity and/or through promoting the internalisation of CFTR from epithelial cell surfaces. Over prolonged periods of exposure this can lead to the formation of mucus characteristic of CF and thus abnormal mucus clearance and/or breathing difficulties in subjects who do not present with overt symptoms of a chronic inflammatory respiratory disorder. The abnormal mucus clearance (or mucus stasis) seen in such subjects mean the respiratory complications observed in CF as discussed above are common in such subjects, e.g. smokers, in particular the complications linked to infection and inflammation of the airways.

Accordingly, in certain embodiments the methods of the invention will further comprise a preceding step in which it is determined that the patient has a defective CFTR ion channel at one or more mucosal surfaces of the patient and/or a mucus sample from the patient is assessed for elevated viscosity and/or the attachment of mucus to the epithelium of one or more mucosal surfaces is assessed. In other embodiments the methods of the invention will further comprise a preceding step in which it is determined that the patient has a condition arising from or associated with a defective CFTR ion channel and/or abnormal mucus which is attached to underlying epithelium. In more specific embodiments, the methods of the invention will further comprise a preceding step in which it is determined that the patient has cystic fibrosis, non-compound CFTR gene mutation heterozygosity, COPD, CB, emphysema, bronchiectasis, asthma and/or chronic sinusitis.

In the case of CF and non-compound CFTR gene mutation heterozygosity, this may for example be by conducting a sweat test, and/or by genetic testing (i.e. by testing for the presence of a mutant CFTR gene, e.g. screening the nucleotide sequences of the patient's CFTR alleles) in combination with the observation and assessment of clinical indicators of CF (in particular mucus viscosity and/or attachment of mucus to the epithelium of mucosal surfaces) and compiling a medical history. In the case of COPD, CB, emphysema, asthma and bronchiectasis this may for example be by measuring lung function with and without bronchodilators, chest imaging and compiling a medical history. In the case of chronic sinusitis this may be by nasal endoscopy and compiling a medical history.

It may be that some of these abovementioned steps are performed to rule out a diagnosis. For instance, a method of the invention may be a method to treat COPD, but this would not necessarily exclude a step in which a patient is assessed for the indicators of CF or a CFTR mutation. Thus, the methods of the invention may further include a preceding step in which it is determined that the patient does not have cystic fibrosis, non-compound CFTR gene mutation heterozygosity, COPD, CB, emphysema, bronchiectasis, asthma and/or chronic sinusitis.

In other embodiments the methods of the invention will further comprise a following step in which the patient's clinical indictors of condition arising from or associated with a defective CFTR ion channel and/or the abnormal attached mucus are assessed and preferably compared to a corresponding assessment made prior to, or earlier in, said treatment in order to determine any changes therein. In more specific embodiments, the methods of the invention will further comprise a following step in which the patient's clinical indictors of CF, non-compound CFTR gene mutation heterozygosity, COPD, CB, emphysema, bronchiectasis, asthma and/or chronic sinusitis (including with respect to the various conditions or complications associated with CF, non-compound CFTR gene mutation heterozygosity, COPD, CB, emphysema, bronchiectasis, asthma and/or chronic sinusitis described above), as appropriate, are assessed and preferably compared to a corresponding assessment made prior to, or earlier in, said treatment in order to determine any changes therein. Parameters relating to the clinical status of a patient with a condition arising from or associated with a defective CFTR ion channel and/or the abnormal attached mucus, e.g. a CF patient and patients with non-compound CFTR gene mutation heterozygosity, COPD, CB, emphysema, bronchiectasis, asthma and/or chronic sinusitis are well known in the art and may be monitored according to known procedures, e.g. in relation to lung performance, lung physiology and measureable signs of inflammation. However, also assessed may be parameters relating to the effect of the alginate oligomers of use in the invention on the mucus and/or secretions in or of the patient, for example attachment of mucus to the epithelium of mucosal surfaces and/or altered mucus properties (e.g. viscosity, in particular sputum viscosity).

In view of the effects of alginate oligomers in which at least 30% of the monomer residues are G residues, when used at the requisite local concentrations on the adherence of mucus to the epithelium of mucosal surfaces, the methods and medical uses of the invention can also be considered to be methods of, or medical uses for, treating the conditions associated with CF in an CF patient, i.e. the complications thereof, which includes preventing, reducing or delaying the development or onset of further conditions associated with CF in a CF patient, or reducing the risk of a CF patient developing or acquiring further conditions associated with CF. This applies also to any of the conditions mentioned or discussed above, and hence analogously to the CF situation above, the invention extends also to the treatment of any condition or complication associated with non-compound CFTR gene mutation heterozygosity, abnormal mucus clearance in the respiratory tract and/or breathing difficulties resulting from a chronic particulate inhalation, and/or a chronic inflammatory respiratory disorder, e.g. COPD, CB, emphysema, bronchiectasis, asthma and/or chronic sinusitis, including preventing, reducing or delaying the development of, or onset of, or risk of further condition. In particular, as explained above a condition associated with CF, or with any of the other conditions mentioned above is a condition which arises as a result of or due to the abnormal mucus characteristic of CF and/or the impaired functioning of the defective CFTR ion channel (i.e. CFTR dysfunction) in such patients.

Such conditions (complications) may be any of those described above or as recited in the following sections. For convenience, in the following such conditions are expressed by reference to CFTR dysfunction-associated conditions, but such terms may be interpreted, where context permits, as a condition (or complication) associated with any of the above-listed conditions, e.g. CF, a non-compound CFTR gene mutation heterozygosity, etc. as listed above. Thus, such conditions (complications) may be CFTR dysfunction-associated respiratory tract conditions (e.g. respiratory tract infections, respiratory tract inflammations, breathing difficulties, respiratory failure and lung remodelling), CFTR dysfunction-associated cardiovascular conditions (e.g. pulmonary hypertension and heart failure); CFTR dysfunction-associated paranasal sinus conditions (e.g. paranasal sinus infection, sinusitis facial pain, headaches, abnormal nasal drainage, nasal polyps); CFTR dysfunction-associated GI conditions (e.g. constipation, bowel obstruction (e.g. meconium ileus in neonatal subjects and intussusception and DIOS in older patients), nutrient malabsorption); CFTR dysfunction-associated pancreatic conditions (e.g. pancreatic duct obstruction, nutrient malabsorption, pancreatic inflammation, pancreatitis (acute and chronic), diabetes); CFTR dysfunction-associated hepatic conditions (e.g. bile duct obstruction, gallstones, liver cirrhosis); and CFTR dysfunction-associated infertility. The treatment of CFTR dysfunction-associated pulmonary, GI, pancreatic and hepatic conditions (e.g. those specified above) is preferred. The present invention is therefore useful prophylactically, since by treating CFTR dysfunction in a patient with an alginate oligomer and helping to restore a more normal mucus phenotype, the development of CFTR dysfunction-associated infections and/or inflammation (most notably in the respiratory tract, GI tract, pancreas and/or liver) may be avoided (i.e. reduced or prevented). It is notable that in the context of the treatment of GI, pancreatic and hepatic conditions associated with CFTR dysfunction, the present treatments represent the fulfilment of an especially long felt need.

In preferred embodiments the alginate oligomers of the invention are used in the treatment of chronic and acute infections and/or inflammations in the lower respiratory tract of patients with a mucosal surface in their respiratory tract that is affected by a lack of functional CFTR ion channels (for example patients with CF, or any of the conditions listed above), e.g. in the bronchi or in the lungs, especially chronic infections and/or inflammation. Expressed alternatively, the alginate oligomers of the invention are used in the treatment of bronchitis or pneumonia in such CF or other patients. Such infections/inflammations (e.g. bronchitis or pneumonia) may commonly be caused by Staphylococcus aureus, Haemophilus influenzae, Pseudomonas aeruginosa, Mycobacterium avium complex, Mycobacterium tuberculosis (the causative agent of pulmonary tuberculosis) and Aspergillus fumigatus although the infections/inflammations may be caused by any infectious agent, e.g. by bacteria, fungus, virus and parasites. In addition to those already mentioned, common infectious agents found in the respiratory tract include, but are not limited to, Chlamydophila pneumonia, Bordetella pertussis, Mycoplasma pneumonia, Moraxella catarrhalis, Legionella pneumophila, Streptococcus pneumonia, Chlamydia psittaci, Coxiella burnetti, rhinovirus, coronavirus, influenza virus, respiratory syncytial virus (RSV), adenovirus, metapneumovirus, parainfluenza virus, Histoplasma capsulatum, Cryptococcus neoformans, Pneumocystis jiroveci, Coccidioides immitis, Toxoplasma gondii, Strongyloides stercoralis, Ascaris lumbricoides, and Plasmodium malariae.

In further preferred embodiments the alginate oligomers of the invention may be used in the treatment of chronic and acute infections and/or inflammations in the upper respiratory tract of patients with a mucosal surface in their respiratory tract that is affected by a lack of functional CFTR ion channels (for example patients with CF or any of the other above-listed conditions), e.g. of the nose, nasal passages, pharynx, larynx and trachea. The treatment of infections and/or inflammations in the trachea of such patients is especially preferred. Expressed alternatively, the alginate oligomers of the invention may be used to treat rhinitis (inflammation of the nasal mucosa), nasopharyngitis (or rhinopharyngitis; inflammation of the nasal mucosa, pharynx, hypopharynx, uvula, and tonsils), pharyngitis (inflammation of the pharynx, hypopharynx, uvula, and tonsils), epiglottitis (or supraglottitis; inflammation of the superior portion of the larynx and supraglottic area), laryngitis (inflammation of the larynx), Iryngotracheitis (inflammation of the larynx, trachea, and subglottic area), tracheitis (inflammation of the trachea and subglottic area) and tonsillitis (inflammation of the tonsils) in such patients (for example patients with CF, COPD or any of the other above-listed conditions. These conditions are sometimes collectively termed upper respiratory tract infections and may be caused by any of the infectious agents mentioned above.

The methods and medical uses of the invention can further be considered as methods of, or medical uses for, increasing the responsiveness of a patient with a condition arising from or associated with a defective CFTR ion channel and/or abnormal mucus which is attached to the underlying epithelium, e.g. a CF patient and patients with non-compound CFTR gene mutation heterozygosity, abnormal mucus clearance in the respiratory tract and/or breathing difficulties resulting from chronic particulate inhalation, and/or a chronic inflammatory respiratory disorder, e.g. COPD, CB, emphysema, bronchiectasis, asthma and/or chronic sinusitis, especially such a patient with a lung infection, to antimicrobial agents, e.g. the antibiotics, antifungals and antivirals recited below. Responsiveness to an antimicrobial is reference to the effects on an infection observed at the patient level for a particular dose of antimicrobial administered in a particular manner. This includes any sign or symptom of the infection observed at the patient level, e.g. microbial load (total or at a specific location), inflammation, fever, microbial toxin levels and general well-being.

As noted above, alginates typically occur as polymers of an average molecular mass of at least 35,000 Daltons, i.e. approximately 175 to approximately 190 monomer residues, although typically much higher and an alginate oligomer according to the present invention may be defined as a material obtained by fractionation (i.e. size reduction) of an alginate polymer, commonly a naturally occurring alginate. An alginate oligomer can be considered to be an alginate of an average molecular weight of less than 35,000 Daltons (i.e. less than approximately 190 or less than approximately 175 monomer residues), in particular an alginate of an average molecular weight of less than 30,000 Daltons (i.e. less than approximately 175 or less than approximately 150 monomer residues) more particularly an average molecular weight of less than 25,000 or 20,000 Daltons (i.e. less than approximately 135 or 125 monomer residues or less than approximately 110 or 100 monomer residues).

Viewed alternatively, an oligomer generally comprises 2 or more units or residues and an alginate oligomer for use according to the invention will typically contain 2 to 100 monomer residues, more typically 3, 4, 5 or 6 to 100, and may contain 2, 3, 4, 5 or 6 to 75, 2, 3, 4, 5 or 6 to 50, 2, 3, 4, 5 or 6 to 40, 2, 3, 4, 5 or 6 to 35 or 2, 3, 4, 5 or 6 to 30 residues. Thus, an alginate oligomer for use according to the invention will typically have an average molecular weight of 350, 550, 700, 900 or 1000 to 20,000 Daltons, 350, 550, 700, 900 or 1000 to 15,000 Daltons, 350, 550, 700, 900 or 1000 to 10,000 Daltons, 350, 550, 700, 900 or 1000 to 8000 Daltons, 350, 550, 700, 900 or 1000 to 7000 Daltons, or 350, 550, 700, 900 or 1000 to 6,000 Daltons.

Alternatively put, the alginate oligomer may have a degree of polymerisation (DP), or a number average degree of polymerisation (DPn) of 2 to 100, preferably 2 to 75, preferably 2 to 50, more preferably 2 to 40, 2 to 35, 2 to 30, 2 to 28, 2 to 25, 2 to 22, 2 to 20, 2 to 18, 2 to 17, 2 to 15 or 2 to 12.

Other representative ranges (whether for the number of residues, DP or DPn) include any one of 3, 4, 5, 6, 7, 8, 9, 10 or 11 to any one of 50, 45, 40, 39, 38, 37, 36, 35, 34, 33, 32, 31, 30, 29, 28, 27, 26, 25, 24, 23, 22, 21, 20, 19, 18, 17, 16, 15, 14, 13 or 12.

Other representative ranges (whether for the number of residues, DP or DPn) include any one of 8, 9, 10, 11, 12, 13, 14 or 15 to any one of 50, 45, 40, 39, 38, 37, 36, 35, 34, 33, 32, 31, 30, 29, 28, 27, 26, 25, 24, 23, 22, 21, 20, 19, 18, 17 or 16.

Other representative ranges (whether for the number of residues, DP or DPn) include any one of 11, 12, 13, 14, 15, 16, 17 or 18 to any one of 50, 45, 40, 39, 38, 37, 36, 35, 34, 33, 32, 31, 30, 29, 28, 27, 26, 25, 24, 23, 22, 21, 20 or 19.

In general terms, an alginate oligomer will, as noted above, contain (or comprise) guluronate or guluronic acid (G) and/or mannuronate or mannuronic acid (M) residues or units. An alginate oligomer according to the invention will preferably be composed solely, or substantially solely (i.e. consist essentially of) uronate/uronic acid residues, more particularly solely or substantially solely of G and M residues or G residues, so long as at least 30% of the monomer residues are G residues. Alternatively expressed, in the alginate oligomer of use in the present invention, at least 80%, more particularly at least 85, 90, 95 or 99% of the monomer residues may be uronate/uronic acid residues, or, more particularly G and M residues or G residues, so long as at least 30% of the monomer residues are G residues. In other words, preferably the alginate oligomer will not comprise other residues or units (e.g. other saccharide residues, or more particularly other uronic acid/uronate residues).

The alginate oligomer is preferably a linear oligomer.

As already indicated, at least 30% of the monomer residues of the alginate oligomer are G residues (i.e. guluronate or guluronic acid). In other words the alginate oligomer will contain at least 30% guluronate (or guluronic acid) residues. Specific embodiments thus include alginate oligomers with (e.g. containing) 30 to 70% G (guluronate) residues or 70 to 100% G (guluronate) residues. Thus, a representative alginate oligomer for use according to the present invention may contain at least 70% G residues (i.e. at least 70% of the monomer residues of the alginate oligomer will be G residues).

Preferably at least 40%, 45%, 50%, 55% or 60%, more particularly at least 70% or 75%, even more particularly at least 80, 85, 90, 91, 92, 93, 94, 95, 96, 97, 98 or 99% of the monomer residues are guluronate. In one embodiment the alginate oligomer may be an oligoguluronate (i.e. a homooligomer of G, or 100% G)

In a further preferred embodiment, the above described alginates of the invention have a primary structure wherein the majority of the G residues are in so called G-blocks. Preferably at least 50%, more preferably at least 70 or 75%, and most preferably at least 80, 85, 90, 92 or 95% of the G residues are in G-blocks. A G block is a contiguous sequence of at least two G residues, preferably at least 3 contiguous G residues, more preferably at least 4 or 5 contiguous G residues, most preferably at least 7 contiguous G residues.

In particular at least 90% of the G residues are linked 1-4 to another G residue. More particularly at least 95%, more preferably at least 98%, and most preferably at least 99% of the G residues of the alginate are linked 1-4 to another G residue.

The alginate oligomer of use in the invention is preferably a 3- to 35-mer, more preferably a 3- to 28-mer, in particular a 4- to 25-mer, e.g. a 5- to 20-mer, especially a 6- to 22-mer, in particular an 8- to 20-mer, especially a 10- to 15-mer, e.g. having a molecular weight in the range 350 to 6400 Daltons or 350 to 6000 Daltons, preferably 550 to 5500 Daltons, preferably 750 to 5000 Daltons, and especially 750 to 4500 Daltons or 2000 to 3000 Daltons or 900 to 3500 Daltons. Other representative alginate oligomers include, as mentioned above, oligomers with 5, 6, 7, 8, 9, 10, 11, 12 or 13 to 50, 45, 40, 35, 28, 25, 22 or 20 residues.

It may be a single compound or it may be a mixture of compounds, e.g. of a range of degrees of polymerization. As noted above, the monomeric residues in the alginate oligomer, may be the same or different and not all need carry electrically charged groups although it is preferred that the majority (e.g. at least 60%, preferably at least 80% more preferably at least 90%) do. It is preferred that a substantial majority, e.g. at least 80%, more preferably at least 90% of the charged groups have the same polarity. In the alginate oligomer, the ratio of hydroxyl groups to charged groups is preferably at least 2:1, more especially at least 3:1.

The alginate oligomer of the invention may have a degree of polymerisation (DP), or a number average degree of polymerisation (DPₙ), of 3-28, 4-25, 6-22, 8-20 or 10-15, or 5-18 or 7-15 or 8-12, especially 10.

The alginate oligomer of the invention may have a degree of polymerisation (DP), or a number average degree of polymerisation (DPₙ), of 3-24, 4-23, 5-22, 6-21, 7-20, 8-19, 9-18, 10-17, 11-16, 12-15 or 13-14 (e.g. 13 or 14).

The alginate oligomer of the invention may have a degree of polymerisation (DP), or a number average degree of polymerisation (DPn), of 4-25, 5-24, 6-23, 7-22, 8-21, 9-20, 10-19, 11-18, 12-17, 13-16, 14-15 (e.g. 14 or 15).

The alginate oligomer of the invention may have a degree of polymerisation (DP), or a number average degree of polymerisation (DPₙ), of 5-26, 6-25, 7-24, 8-23, 9-22, 10-21, 11-20, 12-19, 13-18, 14-17 or 15-16 (e.g. 15 or 16).

The alginate oligomer of the invention may have a degree of polymerisation (DP), or a number average degree of polymerisation (DPₙ), of 4-50, 4-40, 4-35, 4-30, 4-28, 4-26, 4-22, 4-20, 4-18, 4-16 or 4-14.

The alginate oligomer of the invention may have a degree of polymerisation (DP), or a number average degree of polymerisation (DPₙ), of 5-50, 5-40, 5-25, 5-22, 5-20, 5-18, 5-23, 5-20, 5-18, 5-16 or 5-14.

The alginate oligomer of the invention may have a degree of polymerisation (DP), or a number average degree of polymerisation (DPₙ), of 6-50, 6-40, 6-35, 6-30, 6-28, 6-26, 6-24, 6-20, 6-19, 6-18, 6-16 or 6-14.

The alginate oligomer of the invention may have a degree of polymerisation (DP), or a number average degree of polymerisation (DPₙ), of 8-50, 8-40, 8-35, 8-30, 8-28, 8-25, 8-22, 8-20, 8-18, 8-16 or 8-14.

The alginate oligomer of the invention may have a degree of polymerisation (DP), or a number average degree of polymerisation (DPₙ), of 9-50, 9-40, 9-35, 9-30, 9-28, 9-25, 9-22, 9-20, 9-18, 9-16 or 9-14.

The alginate oligomer of the invention may have a degree of polymerisation (DP), or a number average degree of polymerisation (DPₙ), of 10-50, 10-40, 10-35, 10-30, 10-28, 10-25, 10-22, 10-20, 10-18, 10-16 or 10-14.

The alginate oligomer of the invention may have a degree of polymerisation (DP), or a number average degree of polymerisation (DPₙ), of 11-50, 11-40, 11-35, 11-30, 11-28, 11-25, 11-22, 11-20, 11-18, 11-16 or 11-14.

The alginate oligomer of the invention may have a degree of polymerisation (DP), or a number average degree of polymerisation (DPₙ), of 12-50, 12-40, 12-35, 12-30, 12-28, 12-25, 12-22, 12-20, 12-18, 12-16 or 12-14.

The alginate oligomer of the invention may have a degree of polymerisation (DP), or a number average degree of polymerisation (DPₙ), of 13-50, 13-40, 13-35, 13-30, 13-28, 13-25, 13-22, 13-20, 13-18, 13-16 or 13-14.

The alginate oligomer of the invention may have a degree of polymerisation (DP), or a number average degree of polymerisation (DPₙ), of 14-50, 14-40, 14-35, 14-30, 14-28, 14-25, 14-22, 14-20, 14-18, 14-16 or 14-15.

The alginate oligomer of the invention may have a degree of polymerisation (DP), or a number average degree of polymerisation (DPₙ), of 15-50, 15-40, 15-35, 15-30, 15-28, 15-25, 15-22, 15-20, 15-18 or 15-16.

The alginate oligomer of the invention may have a degree of polymerisation (DP), or a number average degree of polymerisation (DPₙ), of 18-50, 18-40, 18-35, 18-30, 18-28, 18-25, 18-22 or 18-20.

Preferably the alginate oligomer of the invention is substantially free, preferably essentially free, of alginate oligomers having a degree of polymerisation outside of the ranges disclosed herein. This may be expressed in terms of the molecular weight distribution of the alginate oligomer of the invention, e.g. the percentage of each mole of the alginate oligomer being used in accordance with the invention which has a DP outside the relevant range. The molecular weight distribution is preferably such that no more than 10%, preferably no more than 9, 8, 7, 6, 5, 4, 3, 2, or 1% mole has a DP of three, two or one higher than the relevant upper limit for DPₙ. Likewise it is preferred that no more than 10%, preferably no more than 9, 8, 7, 6, 5, 4, 3, 2, or 1% mole has a DP below a number three, two or one smaller than the relevant lower limit for DPₙ.

Suitable alginate oligomers are described in WO2007/039754, WO2007/039760, WO 2008/125828, and WO2009/068841, the disclosures of which are explicitly incorporated by reference herein in their entirety.

Representative suitable alginate oligomers have a DPₙ in the range 5 to 30, a guluronate fraction (F_{G}) of at least 0.80, a mannuronate fraction (F_{M}) of no more than 0.20, and at least 95 mole % of DP no more than 25.

Further suitable alginate oligomers have a number average degree of polymerization in the range 7 to 15 (preferably 8 to 12), a guluronate fraction (F_{G}) of at least 0.85 (preferably at least 0.90), a mannuronate fraction (F_{M}) of no more than 0.15 (preferably no more than 0.10), and having at least 95% mole with a degree of polymerization less than 17 (preferably less than 14).

Further suitable alginate oligomers have a number average degree of polymerization in the range 5 to 18 (especially 7 to 15), a guluronate fraction (F_{G}) of at least 0.80 (preferably at least 0.85, especially at least 0.92), a mannuronate fraction (F_{M}) of no more than 0.20 (preferably no more than 0.15, especially no more than 0.08), and having at least 95% mole with a degree of polymerization less than 20 (preferably less than 17).

Further suitable alginate oligomers have a number average degree of polymerization in the range 5 to 18, a guluronate fraction (F_{G}) of at least 0.92, a mannuronate fraction (F_{M}) of no more than 0.08, and having at least 95% mole with a degree of polymerization less than 20.

Further suitable alginate oligomers have a number average degree of polymerization in the range 5 to 18 (preferably 7 to 15, more preferably 8 to 12, especially about 10), a guluronate fraction (F_{G}) of at least 0.80 (preferably at least 0.85, more preferably at least 0.90, especially at least 0.92, most especially at least 0.95), a mannuronate fraction (F_{M}) of no more than 0.20 (preferably no more than 0.15, more preferably no more than 0.10, especially no more than 0.08, most especially no more than 0.05), and having at least 95% mole with a degree of polymerization less than 20 (preferably less than 17, more preferably less than 14).

Further suitable alginate oligomers have a number average degree of polymerization in the range 7 to 15 (preferably 8 to 12), a guluronate fraction (F_{G}) of at least 0.92 (preferably at least 0.95), a mannuronate fraction (F_{M}) of no more than 0.08 (preferably no more than 0.05), and having at least 95% mole with a degree of polymerization less than 17 (preferably less than 14).

Further suitable alginate oligomers have a number average degree of polymerization in the range 5 to 18, a guluronate fraction (F_{G}) of at least 0.80, a mannuronate fraction (F_{M}) of no more than 0.20, and having at least 95% mole with a degree of polymerization less than 20.

Further suitable alginate oligomers have a number average degree of polymerization in the range 7 to 15, a guluronate fraction (F_{G}) of at least 0.85, a mannuronate fraction (F_{M}) of no more than 0.15, and having at least 95% mole with a degree of polymerization less than 17.

Further suitable alginate oligomers have a number average degree of polymerization in the range 7 to 15, a guluronate fraction (F_{G}) of at least 0.92, a mannuronate fraction (F_{M}) of no more than 0.08, and having at least 95% mole with a degree of polymerization less than 17.

Further suitable alginate oligomers have a number average degree of polymerization in the range 5 to 20, a guluronate fraction (F_{G}) of at least 0.85 and a mannuronate fraction (F_{M}) of no more than 0.15.

Further suitable alginate oligomers have a number average degree of polymerization about 13 (e.g. 12, 13 or 14), a guluronate fraction (F_{G}) of at least about 0.80, 0.85, 0.87, 0.88, 0.90 or 0.93 (e.g. 0.92, 0.93 or 0.94) and a corresponding mannuronate fraction (F_{M}) of no more than about 0.20, 0.15, 0.13, 0.12, 0.10, or 0.07 (e.g. 0.08, 0.07 or 0.06).

Further suitable alginate oligomers have a number average degree of polymerization about 21 (e.g. 20, 21 or 22), a guluronate fraction (F_{G}) of at least about 0.80 (e.g. 0.85, 0.87, 0.88, 0.90, 0.92, 0.94 or 0.95) and a corresponding mannuronate fraction (F_{M}) of no more than about 0.20 (e.g. 0.15, 0.13, 0.12, 0.10, 0.08, 0.06, 0.05).

Further suitable alginate oligomers have a number average degree of polymerization about 6 (e.g. 5, 6 or 7), a guluronate fraction (F_{G}) of at least about 0.80 (e.g. 0.85, 0.87, 0.88, 0.90, 0.92, 0.94 or 0.95) and a corresponding mannuronate fraction (F_{M}) of no more than about 0.20 (e.g. 0.15, 0.13, 0.12, 0.10, 0.08, 0.06, 0.05).

It will thus be seen that a particular class of alginate oligomers favoured according to the present invention is alginate oligomers defined as so-called “high G” or “G-block” oligomers i.e. having a high content of G residues or G-blocks (e.g. wherein at least 70% of the monomer residues are G, preferably arranged in G-blocks). However, other types of alginate oligomer may also be used, including in particular oligomers which have a higher content of M residues, including M residues arranged in M-blocks, as long as the oligomer comprises at least 30% G residues, whether arranged singly and/or in G-blocks. Particularly included also are so-called MG-block oligomers, which have an alternating M/G residue structure, as described further below.

Particularly preferred are oligomers wherein at least 70% of the monomer residues in the oligomer are G residues linked 1-4 to another G-residue, or more preferably at least 75%, and most preferably at least 80, 85, 90, 92, 93, 94, 95, 96, 97, 98, 99% of the monomers residues of the oligomer are G residues linked 1-4 to another G residue. This 1-4 linkage of two G residues can be alternatively expressed as a guluronic unit bound to an adjacent guluronic unit.

As noted above, in certain embodiments, any M residues present in the oligomers for use according to the invention may be arranged in M-blocks. For example, in such an embodiment at least 50%, or more particularly at least 70 or 75%, e.g. at least 80, 85, 90 or 95% of the M residues may be in M-blocks. An M block is a contiguous sequence of at least two M residues, preferably at least 3 contiguous M residues, more preferably at least 4 or 5 contiguous M residues, most preferably at least 7 contiguous M residues.

In particular, at least 90% of the M residues may be linked 1-4 to another M residue. More particularly at least 95% at least 98% or at least 99% of the M residues of the alginate may be linked 1-4 to another M residue.

In a still further embodiment, the alginate oligomers of the invention comprise a sequence of alternating M and G residues. A sequence of at least three, preferably at least four, alternating M and G residues represents an MG block. Preferably the alginate oligomers of the invention comprise an MG block. Expressed more specifically, an MG block is a sequence of at least three contiguous residues consisting of G and M residues and wherein each non-terminal (internal) G residue in the contiguous sequence is linked 1-4 and 4-1 to an M residue and each non-terminal (internal) M residue in the contiguous sequence is linked 1-4 and 4-1 to a G residue. Preferably the MG block is at least 5 or 6 contiguous residues, more preferably at least 7 or 8 contiguous residues.

In a further embodiment the minority uronate in the alginate oligomer (i.e. mannuronate or guluronate) is found predominantly in MG blocks. In this embodiment preferably at least 50%, more preferably at least 70 or 75% and most preferably at least 80, 85, 90 or 95% of the minority uronate monomers in the MG block alginate oligomer are present in MG blocks. In another embodiment the alginate oligomer is arranged such that at least 50%, at least 60%, at least 70%, at least 80%, at least 85%, at least 90%, at least 95%, at least 99%, e.g. 100% of the G and M residues in the oligomer are arranged in MG blocks.

In accordance with the above, the MG block containing alginate oligomer will contain at least 30% (or at least 35, 40 or 45% or 50% G) but less than 100% G. Specific embodiments thus include MG block containing alginate oligomers with (e.g. containing) 30 to 70% G (guluronate) residues or 70 to 99% G (guluronate) residues. Thus, a representative MG block containing alginate oligomer for use according to the present invention may contain more than 30%, but less than 70%, G residues (i.e. more than 30%, but less than 70%, of the monomer residues of the MG block alginate oligomer will be G residues).

Preferably more than 30%, more particularly more than 35% or 40%, even more particularly more than 45, 50, 55, 60 or 65%, but in each case less than 70%, of the monomer residues of the MG block containing alginate oligomer are guluronate. Alternatively, less than 70%, more preferably less than 65% or 60%, even more preferably less than 55, 50, 45, 40 or 35%, but in each case more than 30% of the monomer residues of the MG block containing alginate oligomer are guluronate. Any range formed by any combination of these values may be chosen. Therefore for instance the MG block containing alginate oligomer can have e.g. between 35% and 65%, 40% and 60% or 45% and 55% G residues.

In another embodiment the MG block containing alginate oligomer may have approximately equal amounts of G and M residues (e.g. ratios between 65% G/35% M and 35% G/65% M, for instance 60% G/40% M and 40% G/60% M; 55% G/45% M and 45% G/55% M; 53% G/47% M and 47% G/53% M; 51% G/49% M and 49% G/51% M; e.g. about 50% G and about 50% M) and these residues are arranged predominantly, preferably entirely or as completely as possible, in an alternating MG pattern (e.g. at least 50% or at least 60, 70, 80, 85, 90 or 95% or 100% of the M and G residues are in an alternating MG sequence).

In certain embodiments the terminal uronic acid residues of the oligomers of the invention do not have a double bond, especially a double bond situated between the C₄ and C₅ atom. Such oligomers may be described as having saturated terminal uronic acid residues. The skilled man would be able to prepare oligomers with saturated terminal uronic acid residues without undue burden. This may be through the use of production techniques which yield such oligomers, or by converting (saturating) oligomers produced by processes that yield oligomers with unsaturated terminal uronic acid residues.

The alginate oligomer will typically carry a charge and so counter ions for the alginate oligomer may be any physiologically tolerable ion, especially those commonly used for charged drug substances, e.g. sodium, potassium, ammonium, chloride, mesylate, meglumine, etc. Ions which promote alginate gelation e.g. group 2 metal ions may also be used.

While the alginate oligomer may be a synthetic material generated from the polymerisation of appropriate numbers of guluronate and mannuronate residues, the alginate oligomers of use in the invention may conveniently be obtained, produced or derived from natural sources such as those mentioned above, namely natural alginate source materials.

Polysaccharide to oligosaccharide cleavage to produce the alginate oligomer useable according to the present invention may be performed using conventional polysaccharide lysis techniques such as enzymatic digestion and acid hydrolysis. In one favoured embodiment acid hydrolysis is used to prepare the alginate oligomers on the invention. In other embodiments enzymatic digestion is used with an additional processing step(s) to saturate the terminal uronic acids in the oligomers.

Oligomers may then be separated from the polysaccharide breakdown products chromatographically using an ion exchange resin or by fractionated precipitation or solubilisation or filtration. U.S. Pat. No. 6,121,441 and WO 2008/125828, which are explicitly incorporated by reference herein in their entirety, describe a process suitable for preparing the alginate oligomers of use in the invention. Further information and discussion can be found in for example in “Handbooks of Hydrocolloids”, Ed. Phillips and Williams, CRC, Boca Raton, Fla., USA, 2000, which textbook is explicitly incorporated by reference herein in its entirety.

The alginate oligomers may also be chemically modified, including but not limited to modification to add charged groups (such as carboxylated or carboxymethylated glycans) and alginate oligomers modified to alter flexibility (e.g. by periodate oxidation).

Alginate oligomers (for example oligoguluronic acids) suitable for use according to the invention may conveniently be produced by acid hydrolysis of alginic acid from, but not limited to, Laminaria hyperbora and Lessonia nigrescens, dissolution at neutral pH, addition of mineral acid reduce the pH to 3.4 to precipitate the alginate oligomer (oligoguluronic acid), washing with weak acid, resuspension at neutral pH and freeze drying.

The alginates for production of alginate oligomers of the invention can also be obtained directly from suitable bacterial sources e.g. Pseudomonas aeruginosa or Azotobacter vinelandii.

In embodiments where alginate oligomers which have primary structures in which the majority of the G residues are arranged in G-blocks rather than as single residues are required, algal sources are expected to be most suitable on account of the fact that the alginates produced in these organisms tend to have these structures. The bacterial sources may be more suitable for obtaining alginate oligomers of different structures.

The molecular apparatus involved in alginate biosynthesis in Pseudomonas fluorescens and Azotobacter vinelandii has been cloned and characterised (WO 94/09124; Ertesvag, H., et al, Metabolic Engineering, 1999, Vol 1, 262-269; WO 2004/011628; Gimmestad, M., et al (supra); Remminghorst and Rehm, Biotechnology Letters, 2006, Vol 28, 1701-1712; Gimmestad, M. et al, Journal of Bacteriology, 2006, Vol 188(15), 5551-5560) and alginates of tailored primary structures can be readily obtained by manipulating these systems.

The G content of alginates (for example an algal source material) can be increased by epimerisation, for example with mannuronan C-5 epimerases from A. vinelandii or other epimerase enzymes. Thus, for example in vitro epimerisation may be carried out with isolated epimerases from Pseudomonas or Azotobacter, e.g. AlgG from Pseudomonas fluorescens or Azotobacter vinelandii or the AlgE enzymes (AlgE1 to AlgE7) from Azotobacter vinelandii. The use of epimerases from other organisms that have the capability of producing alginate, particularly algae, is also specifically contemplated. The in vitro epimerisation of low G alginates with Azotobacter vinelandii AlgE epimerases is described in detail in Ertesvåg et al (supra) and Strugala et al (Gums and Stabilisers for the Food Industry, 2004, 12, The Royal Society of Chemistry, 84-94).

To obtain G-block containing alginates or alginate oligomers, epimerisation with one or more Azotobacter vinelandii AlgE epimerases other than AlgE4 is preferred as these enzymes are capable of producing G block structures. On the other hand AlgE4 epimerase can be used to create alginates or alginate oligomers with alternating stretches of M/G sequence or primary structures containing single G residue as it has been found that this enzyme seems preferentially to epimerise individual M residues so as to produce single G residues linked to M residues rather than producing G blocks. Particular primary structures can be obtained by using different combinations of these enzymes.

Mutated versions of these enzymes or homologues from other organisms are also specifically contemplated as of use. WO 94/09124 describes recombinant or modified mannuronan C-5 epimerase enzymes (AlgE enzymes) for example encoded by epimerase sequences in which the DNA sequences encoding the different domains or modules of the epimerases have been shuffled or deleted and recombined. Alternatively, mutants of naturally occurring epimerase enzymes, (AlgG or AlgE) may be used, obtained for example by site directed or random mutagenesis of the AlgG or AlgE genes.

A different approach is to create Pseudomonas and Azotobacter organisms that are mutated in some or all of their epimerase genes in such a way that those mutants produce alginates of the required structure for subsequent alginate oligomer production, or even alginate oligomers of the required structure and size (or molecular weight). The generation of a number of Pseudomonas fluorescens organisms with mutated AlgG genes is described in detail in WO 2004/011628 and Gimmestad, M., et al, 2003 (supra). The generation of a number of Azotobacter vinelandii organisms with mutated AlgE genes is disclosed in Gimmestad, M., et al, 2006 (supra).

A further approach is to delete or inactivate the endogenous epimerase genes from an Azotobacter or a Pseudomonas organism and then to introduce one or more exogenous epimerase genes, which may or may not be mutated (i.e. may be wild-type or modified) and the expression of which may be controlled, for example by the use of inducible or other “controllable promoters”. By selecting appropriate combinations of genes, alginates of predetermined primary structure can be produced.

A still further approach would be to introduce some or all of the alginate biosynthesis machinery of Pseudomonas and/or Azotobacter into a non-alginate producing organism (e.g. E. coli) and to induce the production of alginate from these genetically modified organisms.

When these culture-based systems are used, the primary structure of the alginate or alginate oligomer products can be influenced by the culture conditions. It is well within the capabilities of the skilled man to adjust culture parameters such as temperature, osmolarity, nutrient levels/sources and atmospheric parameters in order to manipulate the primary structure of the alginates produced by a particular organism.

References to “G residues/G” and “M residues/M” or to guluronic acid or mannuronic acid, or guluronate or mannuronate are to be read interchangeably as references to guluronic acid/guluronate and mannuronic acid/mannuronate (specifically α-L-guluronic acid/guluronate and β-D-mannuronic acid/mannuronate), and further include derivatives thereof in which one or more available side chains or groups have been modified without resulting in a capacity to treat CF or a CF-associated disorder or condition or a capacity to promote mucus detachment from the epithelium of a mucosal surface that is substantially lower than that of the unmodified oligomer. Common saccharide modifying groups would include acetyl, sulphate, amino, deoxy, alcohol, aldehyde, ketone, ester and anhydro groups. The alginate oligomers may also be chemically modified to add charged groups (such as carboxylated or carboxymethylated glycans), and to alter flexibility (e.g. by periodate oxidation). The skilled man would be aware of still further chemical modifications that can be made to the monosaccharide subunits of oligosaccharides and these can be applied to the alginate oligomers of the invention.

The invention encompasses the use of a single alginate oligomer or a mixture (multiplicity/plurality) of different alginate oligomers. Thus, for example, a combination of different alginate oligomers (e.g. two or more) may be used.

In certain embodiments the local concentration of the alginate oligomer will be 1 to 5.5% w/v, 1 to 5% w/v, 1 to 4.5% w/v, 1 to 4% w/v, 1 to 3.5% w/v, 1 to 3% w/v, 1 to 2.5% w/v, 1 to 2% w/v or 1 to 1.5% w/v.

In certain embodiments the local concentration of the alginate oligomer will be above 1% w/v and equal to or less than 6% w/v, e.g. 1.1 to 6% w/v, 1.1 to 5.5% w/v, 1.1 to 5% w/v, 1.1 to 4.5% w/v, 1.1 to 4% w/v, 1.1 to 3.5% w/v, 1.1 to 3% w/v, 1.1 to 2.5% w/v, 1.1 to 2% w/v or 1.1 to 1.5% w/v.

In certain embodiments the local concentration of the alginate oligomer will be 1.2 to 6% w/v, 1.2 to 5.5% w/v, 1.2 to 5% w/v, 1.2 to 4.5% w/v, 1.2 to 4% w/v, 1.2 to 3.5% w/v, 0.2 to 3% w/v, 1.2 to 2.5% w/v, or 1.2 to 2% w/v or 1.2 to 1.5% w/v

In certain embodiments the local concentration of the alginate oligomer will be 1.5 to 6% w/v, 1.5 to 5.5% w/v, 1.5 to 5% w/v, 1.5 to 4.5% w/v, 1.5 to 4% w/v, 1.5 to 3.5% w/v, 0.5 to 3% w/v, 1.5 to 2.5% w/v, or 1.5 to 2% w/v.

In certain embodiments the local concentration of the alginate oligomer will be 2 to 6% w/v, 2 to 5.5% w/v, 2 to 5% w/v, 2 to 4.5% w/v, 2 to 4% w/v, 2 to 3.5% w/v, 2 to 5% w/v, or 2 to 2.5% w/v.

In certain embodiments the local concentration of the alginate oligomer will be above 2% w/v and equal to or less than 6% w/v, e.g. 2.1 to 6% w/v, 2.1 to 5.5% w/v, 2.1 to 5% w/v, 2.1 to 4.5% w/v, 2.1 to 4% w/v, 2.1 to 3.5% w/v, 2.1 to 3% w/v, or 2.1 to 2.5% w/v.

In certain embodiments the local concentration of the alginate oligomer will be 2.5 to 6% w/v, 2.5 to 5.5% w/v, 2.5 to 5% w/v, 2.5 to 4.5% w/v, 2.5 to 4% w/v, 2.5 to 3.5% w/v or 2.5 to 3% w/v.

In certain embodiments the local concentration of the alginate oligomer will be 3 to 6% w/v, 3 to 5.5% w/v, 3 to 5% w/v, 3 to 4.5% w/v, 3 to 4% w/v or 3 to 3.5% w/v.

In certain embodiments the local concentration of the alginate oligomer will be 3.5 to 6% w/v, 3.5 to 5.5% w/v, 3.5 to 5% w/v, 3.5 to 4.5% w/v or 3.5 to 4% w/v.

In certain embodiments the local concentration of the alginate oligomer will be 4 to 6% w/v, 4 to 5.5% w/v, 4 to 5% w/v or 4 to 4.5% w/v.

In certain embodiments the local concentration of the alginate oligomer will be 4.5 to 6% w/v, 4.5 to 5.5% w/v, 4.5 to 5% w/v.

In certain embodiments the local concentration of the alginate oligomer will be 5.0 to 6% w/v or 5.0 to 5.5% w/v.

In certain embodiments the local concentration of the alginate oligomer will be 5.5 to 6% w/v.

In certain embodiments the local concentration of the alginate oligomer will be equal to or above 1% w/v and less than 2% w/v, e.g. 1 to 1.9% w/v, 1 to 1.8% w/v, 1 to 1.7% w/v, 1 to 1.6% w/v, 1 to 1.5% w/v, 1 to 1.4% w/v, 1 to 1.3% w/v, 1 to 1.2% w/v or 1 to 1.1% w/v.

In certain embodiments the local concentration of the alginate oligomer will be equal to or above 1.1% w/v and less than 2% w/v, e.g. 1.1 to 1.9% w/v, 1.1 to 1.8% w/v, 1.1 to 1.7% w/v, 1.1 to 1.6% w/v, 1.1 to 1.5% w/v, 1.1 to 1.4% w/v, 1.1 to 1.3% w/v, or 1.1 to 1.2% w/v.

In certain embodiments the local concentration of the alginate oligomer will be equal to or above 1.2% w/v and less than 2% w/v, e.g. 1.2 to 1.9% w/v, 1.2 to 1.8% w/v, 1.2 to 1.7% w/v, 1.2 to 1.6% w/v, 1.2 to 1.5% w/v, 1.2 to 1.4% w/v or 1.2 to 1.3% w/v.

“Local concentration” means the concentration of the administered alginate oligomer that is present at the mucosal surface, or more particularly at the mucus layer or coating, e.g. at the lumen/mucus interface, of the target treatment area (i.e. at at least part of the target mucosal surface). Accordingly, “at the mucosal surface”, “at the mucus layer or coating of the mucosal surface” or “at the lumen/mucus interface of the mucosal surface” (which terms are used interchangeably) can be expressed as the “immediate vicinity” of the apical surface of the mucus layer or as “essentially in direct contact” with the apical surface of the mucus layer. Expressed numerically a spatial point less than 1 mm from the apical surface of the mucus layer, e.g. less than 0.5, 0.25, 0.1, 0.05, 0.01, 0.005, 0.001 mm from the apical surface of the mucus layer is at the lumen/mucus interface. In other embodiments the term “local concentration” includes that present within the mucus layer of the mucosal surface at the target treatment area. Without wishing to be bound by theory, the target mucus layer will essentially be fully attached, or partially attached, to the underlying epithelium. The volume under consideration will ultimately be limited by the thickness of the mucus at the target area, which may vary depending on the location of the treatment area, the patient and the severity of their clinical condition, e.g. their CF. In certain embodiments the local concentration is that concentration within the mucus at the lumen/mucus interface. Expressed numerically a spatial point at a depth of less than 1 mm below the apical surface of the mucus layer, e.g. less than 0.5, 0.25, 0.1, 0.05, 0.01, 0.005, 0.001 mm below the apical surface of the mucus layer is at the lumen/mucus interface. In further embodiments local concentration will determined as the concentration (or mean average concentration) present throughout the full depth of the mucus layer at the target treatment area.

“% w/v” (or “percentage weight by volume”) is a commonly used expression of the concentration of a solid solute in a liquid or semi-solid solvent. 1% w/v equates to 1 gram of solid per 100 ml of solvent, 2% w/v equates to 2 g of solid per 100 ml of solvent, and so on. Accordingly local concentration may be expressed as g/100 ml, grams per 100 milliliters, g100 ml⁻¹. 1% w/v also equates to 10 gram of solid per litre of solvent and so the local concentration range of the present invention can be expressed and 10 g/l to 60 g/l. The skilled man would understand that through appropriate scaling calculations, the local concentration range of the present range can be expressed in terms of any SI unit of mass and volume. Conversion into non-standard measures of concentration is also possible and would be routine to the skilled man.

In the context of the present invention “local concentration” will typically amount to the concentration of the alginate oligomer of the invention in the body fluid present at the lumen/mucus interface of the target mucosal surface, the aqueous outer layer of the mucus (e.g. in the case of the respiratory tract, paranasal sinuses and parts of the reproductive system where an air filled lumen is present), or the topical delivery vehicle if so used. As mentioned above in other embodiments the term “local concentration” also includes that present within the mucus layer of the mucosal surface at the target treatment area.

The relevant volume of the solvent/mucus will be determined in part by the size of the target treatment area under consideration. This may be all or part of the respiratory tract, the GI tract, the pancreatic duct, the bile duct, the paranasal sinuses, e.g. those parts recited below, or a subsection thereof. As mentioned above “at the lumen/mucus interface” of the mucosal surface of the target treatment area means a spatial point less than 1 mm from the apical surface of the mucus layer. Within that volume a sufficient mass of alginate must be present to achieve the effective concentration ranges of the present invention.

The skilled man would be able to determine routinely the amount of alginate oligomer he would need to administer to achieve the necessary concentration thereof at the lumen/mucus interface of the mucosal surface at the target treatment area. This amount will vary depending on the location of the target treatment area, the route of administration and dosage form being used and the particular pharmacokinetic factors that are relevant, but the skilled man would be able to consider all the factors and arrive at a suitable dosing regimen. In the case of topical compositions, the composition may simply be formulated to contain the alginate oligomer at the requisite local concentration. Any improvement in any of the symptoms or indicators of the condition being treated in accordance with the invention in a patient (for example CF or any of the other above-mentioned conditions), or e.g. in the various CFTR dysfunction-associated (e.g. CF-associated) disorders or conditions (complications) of CF, or any other condition, displayed by the patient, or any prophylactic or preventative effect in such a patient, can be considered indicative that the appropriate local concentration has been achieved.

Local concentration can be measured directly to ensure appropriate dosing. This may be achieved through sample extraction and analysis or by imaging labelled versions of the alginate oligomer. Suitable sample collection techniques will depend on the target treatment area, but in general can include sputum collection (respiratory tract), swabbing (e.g. nose, mouth and throat, lower GI tract and lower female reproductive tract), mucus biopsy and tissue biopsy, e.g. via an endoscopic procedure. Such procedures include esophagogastroduodenoscopy (oesophagus, stomach and duodenum), enteroscopy (small intestine), colonoscopy, (colon), sigmoidoscopy (large intestine) cholangioscopy (bile and pancreatic ducts), rectoscopy (rectum), anoscopy (anus), proctoscopy (anus and rectum), rhinoscopy (nose/sinus), bronchoscopy (lower respiratory tract), otoscopy (ear), cystoscopy (urinary tract), gynoscopy (female reproductive system), colposcopy (cervix), hysteroscopy (uterus), falloposcopy (fallopian tubes), laparoscopy (abdominal or pelvic cavity). Labelled alginate oligomers may be radioactive or luminescent (e.g. fluorescent). The signals emanating from these labelled alginate oligomers can be detected via appropriate means and quantified and then used to calculate local concentration.

The mucosal surface may be in the respiratory system, e.g. the upper respiratory tract (nose, nasal passages, pharynx larynx and trachea), the paranasal sinuses and the bronchi (primary, secondary and tertiary) and bronchioles of the lower respiratory tract. Preferably the mucosal surface will be in the respiratory tract, preferably the trachea, bronchi and bronchioles.

Inducing mucus detachment from the epithelial cells of the mucosal surfaces of the respiratory system affected by a lack of functional CFTR ion channels is proposed to result in improved mucociliary clearance and improvement in the condition being treated (in particular respiratory tract conditions associated with CF or with any of the other conditions/respiratory tract complications of CF or any of the other conditions, (e.g. respiratory tract infections, respiratory tract inflammations (pneumonia and bronchitis), breathing difficulties, respiratory failure and lung remodelling). The reduction in bacteria and mucus accumulation in the respiratory tract is proposed to reduce or prevent the development of the cardiovascular conditions/cardiovascular complications of CF or other conditions (e.g. pulmonary hypertension and heart failure).

The treatments of the invention are proposed also to improve paranasal sinus conditions/paranasal sinus complications of or associated with CF or with any of the other conditions (e.g. paranasal sinus infection, sinusitis, facial pain, headaches, abnormal nasal drainage, nasal polyps).

The mucosal surface may be in the gastrointestinal tract, e.g. the mouth, the pharynx, the oesophagus, the duodenum and the small intestine (the jejunum and the ileum). The upper GI tract consists of the mouth, pharynx, oesophagus, stomach, and duodenum, and the lower GI tract, consists of the small intestine, the large intestine (the cecum, the colon and the rectum) and the anus. Inducing mucus detachment from the epithelial cells of the mucosal surface of the GI tract affected by a lack of functional CFTR ion channels, especially those in the mouth, the pharynx, the oesophagus, the duodenum, and the small intestine (the jejunum and the ileum) is proposed to result in improvement in CF- or non-compound CFTR gene mutation heterozygosity-associated GI conditions/GI complications of CF or non-compound CFTR gene mutation heterozygosity (e.g. constipation, bowel obstruction (e.g. meconium ileus in neonatal subjects and intussusception and DIOS in older patients), nutrient malabsorption).

The mucosal surface may be in the pancreatic and/or bile ducts. Inducing mucus detachment from the epithelial cells of the mucosal surfaces of the pancreatic and/or bile ducts affected by a lack of functional CFTR ion channels is proposed to result in improvement in CF- or non-compound CFTR gene mutation heterozygosity-associated pancreatic conditions/pancreatic complications of CF or non-compound CFTR gene mutation heterozygosity (e.g. pancreatic duct obstruction, nutrient malabsorption, pancreatic inflammation, pancreatitis (acute and chronic), and diabetes) and/or CF- or non-compound CFTR gene mutation heterozygosity-associated hepatic conditions/hepatic complications of CF (e.g. bile duct obstruction, gallstones and liver cirrhosis).

The mucosal surface may be in the female reproductive system, e.g. the vagina, the cervix, the uterus, the fallopian tubes and the ovaries, preferably the cervix, uterus and the fallopian tubes. The cervix is of particular note. Inducing mucus detachment from the epithelial cells of the mucosal surfaces of the female reproductive system affected by a lack of functional CFTR ion channels is proposed to result in improvement in CF- or non-compound CFTR gene mutation heterozygosity-associated female infertility/female fertility complications of CF or non-compound CFTR gene mutation heterozygosity.

The mucosal surface may be in the male reproductive system, e.g. the testes, the epididymis, the vas deferens, the accessory glands, the seminal vesicles, the prostate gland and the bulbourethral gland. The epididymis and the vas deferens are of particular note. Inducing mucus detachment from the epithelial cells of the mucosal surfaces of the male reproductive system affected by a lack of functional CFTR ion channels is proposed to result in improvement in CF- or non-compound CFTR gene mutation heterozygosity-associated male infertility/male fertility complications of CF or non-compound CFTR gene mutation heterozygosity.

The experimental work described in the Examples have shown that certain local concentrations of alginate oligomers in which at least 30% of the monomer residues are G residues are capable of promoting the conversion of the abnormal phenotype of a mucosal surface affected by a lack of functional CFTR ion channels, and more specifically a mucosal surface from a CF patient, to a phenotype more closely resembling that of a healthy mucosal surface, i.e. a mucosal surface from a subject that is not affected by a lack of functional CFTR ion channels (e.g. a non-CF subject). This is in part believed to be due to the observed detachment of the mucus layer from the mucosal surface affected by a lack of functional CFTR ion channels (e.g. a CF mucosal surface) upon exposure of the mucosal surface to alginate oligomers at certain local concentrations. A mucus layer from a mucosal surface affected by a lack of functional CFTR ion channels that has been detached in accordance with the treatments of the invention is proposed to behave substantially analogously to a normal healthy mucus layer that is not affected by a lack of functional CFTR ion channels and therefore respond to the body's mucus clearance/handling systems in much the same way as the mucus of a healthy subject (e.g. a non-CF subject).

This is expected to result in the alleviation of the condition suffered by the patient undergoing treatment, and/or of any complication or disorder etc. associated with the condition, and/or the prevention of the onset of any further condition or disorder or complication associated with the condition, e.g. as discussed above.

In the particular case of CF this is expected to result in alleviation of the CF-associated disorders or conditions (complications of CF) suffered by the CF patient undergoing treatment and/or prevention of the onset of further CF-associated disorders or conditions (complications of CF), e.g. those discussed above.

In view of this, full (or complete) detachment within the treatment area might not be necessary to give noticeable improvement in a patient's (e.g. a CF patient's) conditions or disorders. As such, in certain embodiments the detachment may be partial. Partial detachment in accordance with the invention can be considered to be that extent of detachment that is therapeutically effective. Any improvement in any of the symptoms or indicators of a condition (for example CF), e.g. the various CF-associated disorders or conditions (complications of CF) displayed by a CF patient, or any complication or symptom or condition associated with any other CFTR dysfunction-related condition in the patient undergoing treatment, or any prophylactic or preventative effect in the patient, can be considered indicative that therapeutically effective detachment has been achieved.

Expressed numerically the mucus layer at the target treatment area is detached over at least 40% of that area, e.g. at least 50, 60, 70, 75, 80, 85, 90, 95, 96, 97, 98, 99% of that area. Preferably the mucus layer at the target treatment area is detached over about 100% of that area.

Detachment may also be viewed as a reduction in mucus adhesiveness or a loosening in the interaction between the mucus layer and the underlying epithelial cells. These properties are thought to result from a variety of chemical, e.g. intra- and inter-molecular, bonds which occur between the mucus components (e.g. mucin) and the epithelial cells. Partial detachment can therefore be considered to be a reduction in mucus adhesiveness/loosening in mucus-epithelium interaction of at least 40%, e.g. at least 50, 60, 70, 75, 80, 85, 90, 95, 96, 97, 98, 99% or about 100%.

The extent of detachment can be measured by any convenient means, e.g. microscopically. Gustafsson et al (supra), which is explicitly incorporated by reference herein in its entirety, provides further details on confocal, transmission electron, and optical (bright field) microscopy techniques for assessing the thickness of mucus layers and attachment/detachment from the underlying epithelium. With suitable calibration the extent of detachment can further be correlated to reduction in mucus adhesiveness/loosening in mucus-epithelium interaction.

As shown in the Examples, a portion of the mucosal surface of the treatment area can be extracted, e.g. by biopsy during endoscopy, and the extent of the detachment of the mucus layer can be measured by measuring mucus thickness before and after a standardised aspiration procedure. Repeating this procedure at a plurality of different locations allows the assessment of the degree of detachment over a certain area. In these, and equivalent, embodiments mucus detachment can also be expressed in terms of average (e.g. mean) mucus thickness over the treatment area following aspiration. Preferably partial detachment will results in a reduction in average (e.g. mean) mucus thickness over the treatment area of at least 40%, e.g. at least 50, 60, 70, 75, 80, 85, 90, 95, 96, 97, 98, 99% or about 100% following aspiration. Depending on the technique used to aspirate the mucus layer it might not be possible to completely remove the mucus layer even though detachment is total (such as would be the case in a sample from a healthy subject or more specifically a mucosal surface which is not affected by a lack of functional CFTR, e.g. a non-CF sample). In such instances 100% should be construed as the maximum thickness reduction that is observed in the experimental context. Maximum thickness reduction can be determined by suitable internal experimental controls.

Mucus attachment can also be recorded and observed by video microscopy where mucus should be normally moved by cilia or by controlled aspiration. The mucus can be visualized by different staining including charcoal and dyes.

It is further apparent from the experimental work of the Examples that the treatments of the present invention do not substantially alter the thickness of the mucus layer at the treatment area. Such a feature is proposed to be an additional benefit to the proposed treatments. As discussed above, many of conditions arising from or associated with a defective CFTR ion channel and/or the abnormal attached mucus, e.g. disorders or conditions associated with CF (complications of CF), are characterised by complete or partial obstruction of a lumen with mucus or thickened exocrine sections (e.g. the lung, GI, liver and pancreatic conditions). A treatment that does not increase mucus thickness is therefore not expected to exacerbate these conditions by causing further narrowing/occlusion of an already narrowed lumen.

The methods and medical uses of the present invention can therefore be considered to be methods and medical uses that may be effected without substantial thickening of the mucus coating of the mucosal surface to which the alginate oligomer has been administered (the treatment area). “Without substantial thickening” may be expressed numerically as a less than 40% increase in thickness, e.g. less than 30, 20, 15, 10, 9, 8, 7, 6, 5, 4, 3, 2, or 1% increase in thickness upon administration of the alginate oligomer to the treatment area. Preferably there is substantially no change to the thickness of the mucus layer at the treatment area. Mucus thickness may be measured before, after and during treatment with alginate oligomers as described above.

“Treatment” when used in relation to the treatment of a condition arising from or associated with a defective CFTR ion channel and/or the abnormal attached mucus in accordance with the invention is used broadly herein to include any therapeutic effect, i.e. any beneficial effect on said condition or symptom or indicator thereof. Such conditions include not only CF, non-compound CFTR gene mutation heterozygosity, abnormal mucus clearance in the respiratory tract and/or breathing difficulties resulting from chronic particulate inhalation, and/or a chronic inflammatory respiratory disorder, e.g. COPD, CB, emphysema, bronchiectasis, asthma and/or chronic sinusitis, but also conditions or disorders associated with any of these conditions. In this section a reference to a condition or disorder associated with CF, non-compound CFTR gene mutation heterozygosity, abnormal mucus clearance in the respiratory tract and/or breathing difficulties resulting from chronic particulate inhalation, and/or a chronic inflammatory respiratory disorder, e.g. COPD, CB, emphysema, bronchiectasis, asthma and/or chronic sinusitis is interchangeable with a reference to a complication of CF, non-compound CFTR gene mutation heterozygosity, abnormal mucus clearance in the respiratory tract and/or breathing difficulties resulting from chronic particulate inhalation, and/or a chronic inflammatory respiratory disorder, e.g. COPD, CB, emphysema, bronchiectasis, asthma and/or chronic sinusitis.

Specifically in the context of CF and non-compound CFTR gene mutation heterozygosity, because these diseases are genetic diseases which are characterised in each patient by the unique collection of CF- and non-compound CFTR gene mutation heterozygosity-associated disorders and conditions displayed by the patient at the time of receiving the treatments of the invention, the terms “treatment of CF” and “treatment of non-compound CFTR gene mutation heterozygosity” can be considered to be the treatment of any or all of the disorders and conditions of the patient or the treatment of a subset thereof.

Thus, although the invention does not address correction of the underlying genetic defect of CF or non-compound CFTR gene mutation heterozygosity, it relates to treatment of the effects in the body which arise from the defect, e.g. an alleviation of the effects thereof, e.g. effects arising from the abnormal mucus, and includes the treatment of an associated disorder or condition and also an improvement in the clinical effects of the disorder or condition or overall well-being of the subject. In this context, a “cure” of CF or non-compound CFTR gene mutation heterozygosity would amount to complete alleviation of the various CF- or non-compound CFTR gene mutation heterozygosity-associated disorders and conditions displayed by the patient at the time of receiving the treatments of the invention; however the genetic basis for the disease (the CFTR mutation) would still remain. Nonetheless, the invention does not require such a “cure” and as noted above, includes an improvement in any effect which the CF or non-compound CFTR gene mutation heterozygosity has on the body. Thus included, for example, is an improvement in any symptom or sign of a CF- or non-compound CFTR gene mutation heterozygosity-associated disorder or condition, or in any clinically accepted indicator of a CF- or non-compound CFTR gene mutation heterozygosity-associated disorder or condition in the patient (for example, increasing mucociliary clearance in the lungs, increased responsiveness of lung infections to antibiotics, reduced incidence of constipation or improvement in nutrient absorption). In the presently claimed treatments it may be that a pre-existing CF- or non-compound CFTR gene mutation heterozygosity-associated disorder or condition is not fully eradicated or the onset of a new CF- or non-compound CFTR gene mutation heterozygosity-associated disorder or condition is not completely halted, but the treatments are sufficient to inhibit these processes to such an extent that the target CF- or non-compound CFTR gene mutation heterozygosity-associated disorder or condition is fully resolved, or at least resolved to some extent, preferably to an extent acceptable to the subject. Treatment thus includes both curative and palliative therapy, e.g. of a pre-existing or diagnosed CF- or non-compound CFTR gene mutation heterozygosity-associated disorder or condition, i.e. a reactionary treatment.

“Prevention”, when used in relation to the treatment of a condition arising from or associated with a defective CFTR ion channel and/or the abnormal attached mucus in accordance with the invention, is used broadly herein to include any prophylactic or preventative effect in the patient against said condition. Such conditions include not only CF, non-compound CFTR gene mutation heterozygosity, abnormal mucus clearance in the respiratory tract and/or breathing difficulties resulting from chronic particulate inhalation, and/or a chronic inflammatory respiratory disorder, e.g. COPD, CB, emphysema, bronchiectasis, asthma and/or chronic sinusitis, but also conditions or disorders associated therewith. In this section a reference to a condition or disorder associated with CF, non-compound CFTR gene mutation heterozygosity, abnormal mucus clearance in the respiratory tract and/or breathing difficulties resulting from chronic particulate inhalation, and/or a chronic inflammatory respiratory disorder, e.g. COPD, CB, emphysema, bronchiectasis, asthma and/or chronic sinusitis is interchangeable with a reference to a complication of CF or any said condition. “Prevention” thus, in general terms, includes delaying, limiting, reducing or preventing an effect of said condition or complication, or one or more symptoms or indications thereof, in a patient or the onset of said condition or complication, or one or more symptoms or indications thereof, for example relative to the condition, complication, symptom or indication thereof prior to the prophylactic treatment.

It will be understood of course that CF and non-compound CFTR gene mutation heterozygosity in the sense of the underlying genetic defect cannot be prevented by the present invention and this is not included. “Prevention” in these contexts thus relates to preventing an effect in the body which arises as a result of the underlying genetic defect, or as a result of the abnormal mucus.

Specifically in the context of CF and non-compound CFTR gene mutation heterozygosity, because these diseases are genetic diseases which are characterised in each patient by the unique collection of CF- or non-compound CFTR gene mutation heterozygosity-associated disorders and conditions displayed by the patient at the time of receiving the treatments of the invention, the term “prevention of CF or non-compound CFTR gene mutation heterozygosity or a CF- or non-compound CFTR gene mutation heterozygosity-associated disorder or condition” can be considered to be the prevention of any CF- or non-compound CFTR gene mutation heterozygosity-associated disorder or condition that the patient has yet to acquire or which the patient has acquired previously but has overcome prior to receiving the claimed treatments.

Prophylaxis explicitly includes both absolute prevention of occurrence or development of an effect of a condition arising from or associated with a defective CFTR ion channel and/or the abnormal attached mucus, as defined above, or symptom or indication thereof, and any delay in the onset or development of an effect of a condition arising from or associated with a defective CFTR ion channel and/or the abnormal attached mucus, as defined above, or symptom or indication thereof, or reduction or limitation of the development or progression of a condition arising from or associated with a defective CFTR ion channel and/or the abnormal attached mucus, as defined above, or symptom or indication thereof. The preventative treatments can also be considered as treatments that reduce the risk of a patient acquiring or developing a condition arising from or associated with a defective CFTR ion channel and/or the abnormal attached mucus, as defined above, or symptom or indication thereof.

The terms “patient with CF”, “patient suffering from CF”, “patient having CF” and “CF patient” are considered to be equivalent and are used interchangeably herein. Corresponding terms directed to any of the other conditions arising from or associated with a defective CFTR ion channel and/or the abnormal mucus which is attached to underlying epithelium mentioned herein are used similarly.

In one embodiment of the invention the alginate oligomers as herein defined may be used in the methods or uses of the invention in conjunction or combination with a further pharmaceutical for the treatment of CF or CF-associated disorders or conditions/complications of CF (hereinafter “further CF pharmaceutical”). Such pharmaceutical may also be considered as being for use, inter alia, in the treatment of non-compound CFTR gene mutation heterozygosity, abnormal mucus clearance in the respiratory tract and/or breathing difficulties resulting from chronic particulate inhalation, and/or a chronic inflammatory respiratory disorder, e.g. COPD, CB, emphysema, bronchiectasis, asthma and/or chronic sinusitis, conditions associated therewith or complications thereof.

The further CF or other pharmaceutical (i.e. further therapeutically active agent) may be an antibiotic, an antifungal, an antiviral, an immunostimulatory agent, a corticosteroid, a non-steroidal anti-inflammatory drug (NSAID), a bronchodilator, a digestive enzyme supplement, an oral antidiabetic drug, an injectable antidiabetic drug or a mucus viscosity-reducing agent (i.e. an agent which reduces the viscosity of mucus and which terms are used interchangeably with the term “mucolytic agent”).

The antibiotic may be selected from the aminoglycosides (e.g. amikacin, gentamicin, kanamycin, neomycin, netilmicin, streptomycin, tobramycin); the 6-lactams (e.g. the carbecephems (e.g. loracarbef); the 1st generation cephalosporins (e.g. cefadroxil, cefazolin, cephalexin); 2nd generation cephalosporins (e.g. cefaclor, cefamandole, cephalexin, cefoxitin, cefprozil, cefuroxime); 3rd generation cephalosporins (e.g. cefixime, cefdinir, cefditoren, cefoperazone, cefotaxime, cefpodoxime, ceftazidime, ceftibuten, ceftizoxime, ceftriaxone); 4th generation cephalosporins (e.g. cefepime); the monobactams (e.g. aztreonam); the macrolides (e.g. azithromycin, clarithromycin, dirithromycin, erythromycin, troleandomycin); the monobactams (e.g. aztreonam); the penicillins (e.g. amoxicillin, ampicillin, carbenicillin, cloxacillin, dicloxacillin, nafcillin, oxacillin, penicillin G, penicillin V, piperacillin, ticarcillin); the polypeptide antibiotics (e.g. bacitracin, colistin, polymyxin B); the quinolones (e.g. ciprofloxacin, enoxacin, gatifloxacin, levofloxacin, lomefloxacin, moxifloxacin, norfloxacin, ofloxacin, trovafloxacin); the sulfonamides (e.g. mafenide, sulfacetamide, sulfamethizole, sulfasalazine, sulfisoxazole, trimethoprim-sulfamethoxazole); the tetracyclines (e.g. demeclocycline, doxycycline, minocycline, oxytetracycline, tetracycline); the glycylcyclines (e.g. tigecycline); the carbapenems (e.g. imipenem, meropenem, ertapenem, doripenem, panipenem/betamipron, biapenem, PZ-601); other antibiotics include chloramphenicol; clindamycin, ethambutol; fosfomycin; isoniazid; linezolid; metronidazole; nitrofurantoin; pyrazinamide; quinupristin/dalfopristin; rifampin; spectinomycin; and vancomycin.

More preferably the antibiotic is selected from amikacin, gentamicin, kanamycin, neomycin, netilmicin, streptomycin, tobramycin, cefixime, cefdinir, cefditoren, cefoperazone, cefotaxime, cefpodoxime, ceftazidime, ceftibuten, ceftizoxime, ceftriaxone, cefepime, aztreonam, amoxicillin, ampicillin, carbenicillin, cloxacillin, dicloxacillin, nafcillin, oxacillin, penicillin G, penicillin V, piperacillin, ticarcillin, ciprofloxacin, enoxacin, gatifloxacin, levofloxacin, lomefloxacin, moxifloxacin, norfloxacin, ofloxacin, trovafloxacin, azithromycin, clarithromycin, dirithromycin, erythromycin, roxithromycin, telithromycin, CarbomycinA, josamycin, kitasamycin, midecamicine, oleandomycin, spiramycin, troleandromycin, tylosin, imipenem, meropenem, ertapenem, doripenem, panipenem/betamipron, biapenem, PZ-601, bacitracin, colistin, polymyxin B, demeclocycline, doxycycline, minocycline, oxytetracycline and tetracycline.

More preferably the antibiotic is selected from aztreonam, ciprofloxacin, gentamicin, tobramycin, amoxicillin, colistin, ceftazidime, azithromycin, clarithromycin, dirithromycin, erythromycin, roxithromycin, spiramycin, oxytetracycline, and imipenem.

In particularly preferred embodiments the antibiotic is selected from aztreonam, ciprofloxacin, gentamicin, tobramycin, amoxicillin, colistin and ceftazidime.

Representative antifungals include, but are not limited to the polyenes (e.g. natamycin, rimocidin, filipin, nystatin, amphotericin B, candicin; the imidazoles (e.g. miconazole, ketoconazole, clotrimazole, econazole, bifonazole, butoconazole, fenticonazole, isoconazole, oxiconazole, sertaconazole, sulconazole, tioconazole); the triazoles (e.g. fluconazole, itraconazole, isavuconazole, ravuconazole, posaconazole, voriconazole, terconazole); the allylamines (e.g. terbinafine, amorolfine, naftifine, butenafine); and the echinocandins (e.g. anidulafungin, caspofungin, micafungin).

Representative antivirals include, but are not limited to abacavir, acyclovir, adefovir, amantadine, amprenavir, arbidol, atazanavir, atripla, boceprevir, cidofovir, combivir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, nelfinavir, nevirapine, nexavir, oseltamivir, penciclovir, peramivir, pleconaril, podophyllotoxin, raltegravir, ribavirin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, and zidovudine.

Representative immunostimulatory agents include, but are not limited to cytokines e.g. TNF, IL-1, IL-6, IL-8 and immunostimulatory alginates, such as high M-content alginates as described for example in U.S. Pat. No. 5,169,840, WO91/11205 and WO03/045402 which are explicitly incorporated by reference herein in their entirety, but including any alginate with immunostimulatory properties.

Representative NSAIDs include, but are not limited to, the salicylates (e.g. aspirin (acetylsalicylic acid), choline magnesium trisalicylate, diflunisal, salsalate, the propionic acid derivatives (e.g. ibuprofen, dexibuprofen, dexketoprofen, fenoprofen, flurbiprofen, ketoprofen, loxoprofen, naproxen, oxaprozin), the acetic acid derivatives (e.g. aceclofenac, diclofenac, etodolac, indomethacin, ketorolac, nabumetone, tolmetin, sulindac), the enolic acid derivatives (e.g. droxicam, isoxicam, lornoxicam, meloxicam, piroxicam, tenoxicam), the anthranilic acid derivatives (e.g. flufenamic acid, meclofenamic acid, mefenamic acid, tolfenamic acid) and the selective COX-2 inhibitors (Coxibs; e.g. celecoxib, etoricoxib, lumiracoxib, parecoxib, rofecoxib, valdecoxib). The propionic acid derivatives (e.g. ibuprofen, dexibuprofen, dexketoprofen, fenoprofen, flurbiprofen, ketoprofen, loxoprofen, naproxen, oxaprozin) are preferred, ibuprofen being most preferred.

As used herein, the terms “mucolytic agent” and “mucus viscosity reducing agent” are intended to encompass agents which reduce the intrinsic viscosity of mucus and agents which reduce the attachment of mucus to underlying epithelium, in particular agents which directly or indirectly disrupt the molecular interactions within or between the components of mucus, agents which affect the hydration of mucus and agents which modulate the ionic microenvironment of the mucosal epithelium (particularly the levels of divalent cations, e.g. calcium). Representative examples of suitable mucus viscosity reducing agents include but are not limited to a nucleic acid cleaving enzyme (e.g. a DNase such as DNase I or dornase alfa), hypertonic saline, gelsolin, a thiol reducing agent, an acetylcysteine, an uncharged low molecular weight polysaccharide (e.g. dextran, mannitol), arginine (or other nitric oxide precursors or synthesis stimulators), an agonist of the P2Y2 subtype of purinergic receptors (e.g. denufosol) or an anionic polyamino acid (e.g. poly ASP or poly GLU). Ambroxol, bromhexine, carbocisteine, domiodol, eprazinone, erdosteine, letosteine, mesna, neltenexine, sobrerol, stepronin, tiopronin are specific mucolytics of note. DNase I and hypertonic saline are preferred.

Representative examples of suitable bronchodilators include but are not limited to the β2 agonists (e.g. the short-acting β2 agonists (e.g. pirbuterol, epinephrine, salbutamol, levosalbutamol, clenbuterol, terbutaline, procaterol, metaproterenol, fenoterol, bitolterol mesylate, ritodrine, isoprenaline); the long-acting β2 agonists (e.g. salmeterol, formoterol, bambuterol, clenbuterol); and the ultra-long-acting β2 agonists (e.g. indacaterol)), the anticholinergics (e.g. ipratropium, oxitropium, tiotropium) and theophylline.

Representative examples of suitable corticosteroids include but are not limited to prednisone, flunisolide, triamcinolone, fluticasone, budesonide, mometasone, beclomethasone, amcinonide, budesonide, desonide, fluocinonide, fluocinolone, halcinonide, hydrocortisone, cortisone, tixocortol, prednisolone, methylprednisolone, prednisone, betamethasone, dexamethasone, fluocortolone, aclometasone, prednicarbate, clobetasone, clobetasol, and fluprednidene.

Representative examples of suitable digestive enzyme supplements include but are not limited to pancrelipase (a mixture of pancreatic lipases, amylases, and chymotrypsin), pancreatin (a mixture of pancreatic lipases, amylases, and trypsin) or one or more lipases (e.g. bile salt dependent lipase, pancreatic lipase, gastric lipase, pancreatic lipase related protein 1, pancreatic lipase related protein 2, lingual lipase), proteases (e.g. pepsin, trypsin and chymotrypsin) and amylases (e.g. α-amylase, β-amylase, γ-amylase). These enzymes may be plant enzymes or animal enzymes, including human. These enzymes may be obtained from natural sources or prepared by molecular biology techniques.

Representative examples of suitable oral antidiabetic drugs include, but are not limited to, the sulfonylureas (e.g. carbutamide, acetohexamide, chlorpropamide, tolbutamide, glipizide, gliclazide, glibenclamide, glibornuride, gliquidone, glisoxepide, glyclopyramide, glimepiride), the biguanides (e.g. metformin, phenformin, buformin, proguanil), the thiazolidinediones (e.g. rosiglitazone, pioglitazone, troglitazone), the alpha-glucosidase inhibitors (e.g. acarbose, miglitol, voglibose), the meglitinides (e.g. nateglinide, repaglinide, mitiglinide), and the glycosurics (e.g. dapagliflozin, ganagliflozin, ipragliflozin, tofogliflozin, empagliflozin, sergliflozin etabonate, remogliflozin etabonate).

Representative examples of suitable injectable antidiabetic drugs include, but are not limited to, insulin and its analoges (e.g. insulin lispro, insulin aspart, insulin glulisine, insulin zinc, isophane insulin, insulin glargine, insulin detemir) and the incretin mimetics (e.g. the glucagon-like peptide (GLP) agonists, e.g. exenatide, liraglutide, and taspoglutide; and the dipeptidyl peptidase-4 (DPP-4) inhibitors, e.g. vildagliptin, sitagliptin, saxagliptin, linagliptin, allogliptin and septagliptin).

The further CF pharmaceutical may conveniently be applied before, simultaneously with or following the alginate oligomer. Conveniently the further CF pharmaceutical is applied at substantially the same time as the alginate oligomer or afterwards. In other embodiments the further CF pharmaceutical may conveniently be applied or administered before the alginate oligomer. The further CF pharmaceutical can also be given (e.g. administered or delivered) repeatedly at time points appropriate for the agent used. The skilled person is able to devise a suitable dosage regimen. In long term treatments the alginate oligomer can also be used repeatedly. The alginate oligomer can be applied as frequently as the further CF pharmaceutical, or more or less frequently. The frequency required may depend on the location of the mucosal surface to which the alginate oligomer is administered and also the overall nature of the clinical condition, (e.g. CF) displayed by the particular patient undergoing treatment.

The alginate oligomers proposed for use according to the invention and the further CF pharmaceutical (or further therapeutically active agent), may for example be administered together, in a single pharmaceutical formulation or composition, or separately (i.e. separate, sequential or simultaneous administration). Thus, the alginate oligomers of the invention and the further CF pharmaceutical may be combined, e.g. in a pharmaceutical kit or as a combined (“combination”) product.

The invention therefore also provides products (e.g. a pharmaceutical kit or a combined (“combination”) product) or compositions (e.g. a pharmaceutical composition) wherein the product or composition comprises an alginate oligomer as herein defined and a further CF pharmaceutical (or further therapeutically active agent), e.g. those described above. Combinations comprising an alginate oligomer as herein defined and an antibiotic, an antifungal, an NSAID, a bronchodilator, a corticosteroid and/or a mucus viscosity reducing agent are preferred. Combinations comprising an alginate oligomer as herein defined and an antibiotic, an antifungal and/or a mucus viscosity reducing agent are especially preferred. Such pharmaceutical products and pharmaceutical compositions are preferably adapted for use in the medical methods of the invention.

The use of alginate oligomers as herein defined to manufacture such pharmaceutical products and pharmaceutical compositions for use in the medical methods of the invention is also contemplated.

The alginate oligomers of the invention may be administered to the subject in any convenient form or by any convenient means in order to achieve the requisite local concentration at the mucosal surface of the target treatment area, e.g. by topical, enteral (e.g. oral, buccal, sublingual, rectal), parenteral (e.g. intrahepatic, intrapancreatic) or by inhalation (including nasal inhalation). Preferably the alginate will be administered by enteral routes or by inhalation. Topical administration to parts of the female reproductive system (e.g. the vagina and the cervix) may also be convenient. That alginate oligomers may be administered via many different routes is an advantage over currently available CF pharmaceuticals as relatively straightforward treatment of liver and pancreas complications is made possible.

The skilled man will be able to formulate the alginate oligomers of the invention into pharmaceutical compositions that are adapted for these routes of administration according to any of the conventional methods known in the art and widely described in the literature.

The present invention therefore also provides a pharmaceutical composition for use in any of the above-mentioned methods or uses comprising an alginate oligomer as defined herein, together with at least one pharmaceutically acceptable carrier, diluent or excipient, preferably in an amount sufficient to achieve the requisite local concentration at the mucosal surface of the target treatment area. This composition may also comprise other therapeutic agents as described above.

More specifically, the alginate oligomers of the invention may be incorporated, optionally together with other active agents, with one or more conventional carriers, diluents and/or excipients, to produce conventional galenic preparations such as tablets, pills, powders (e.g. inhalable powders, including dry inhalable powders), lozenges, sachets, cachets, elixirs, suspensions, emulsions, solutions, syrups, aerosols (as a solid or in a liquid medium), sprays (e.g. nasal sprays), compositions for use in nebulisers, ointments, creams, salves, soft and hard gelatine capsules, suppositories, pessaries, sterile injectable solutions, sterile packaged powders, and the like. Enteric coated solid or liquid compositions, sterile inhalable and sterile injectable compositions are of particular note.

Examples of suitable carriers, excipients, and diluents are lactose, dextrose, sucrose, sorbitol, mannitol, starches, gum acacia, calcium phosphate, inert alginate polymers, tragacanth, gelatine, calcium silicate, microcrystalline cellulose, polyvinylpyrrolidone, cellulose, water syrup, water, water/ethanol, water/glycol, water/polyethylene, hypertonic salt water, glycol, propylene glycol, methyl cellulose, methylhydroxybenzoates, propyl hydroxybenzoates, talc, magnesium stearate, mineral oil or fatty substances such as hard fat or suitable mixtures thereof. Excipients and diluents of note are mannitol and hypertonic salt water (saline).

The compositions may additionally include lubricating agents, wetting agents, emulsifying agents, suspending agents, preserving agents, sweetening agents, flavouring agents, and the like. Additional therapeutically active agents may be included in the pharmaceutical compositions, as discussed above in relation to combination therapies above.

Parenterally administrable forms, e.g. solutions suitable for delivery via the intrahepatic or intrapancreatic routes mentioned above, should be sterile and free from physiologically unacceptable agents, and should have low osmolarity to minimize irritation or other adverse effects upon administration and thus solutions should preferably be isotonic or slightly hypertonic, e.g. hypertonic salt water (saline). Suitable vehicles include aqueous vehicles customarily used for administering parenteral solutions such as sterile water for injection, Sodium Chloride Injection, Ringer's Injection, Dextrose Injection, Dextrose and Sodium Chloride Injection, Lactated Ringer's Injection and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975)), which is explicitly incorporated by reference herein in its entirety. The solutions can contain preservatives, antimicrobial agents, buffers and antioxidants conventionally used for parenteral solutions, excipients and other additives which are compatible with the biopolymers and which will not interfere with the manufacture, storage or use of products.

Simple sterile solutions of alginate oligomers or simple sterile liquid compositions comprising alginate oligomers may be especially convenient for use during surgical procedures and for delivery to the lungs, e.g. by nebuliser, or to the paranasal sinuses, e.g. by a nasal spray device.

Solid or liquid formulations of the alginate oligomer may be provided with an enteric coating that prevents degradation in the stomach and/or other parts of the upper GI tract but permits degradation in the lower GI tract, e.g. the small intestine. Such coatings are routinely prepared from polymers including fatty acids, waxes, shellac, plastics, and plant fibres. Specific examples thereof include but are not limited to methyl acrylate-methacrylic acid copolymers, methyl methacrylate-methacrylic acid copolymers, cellulose acetate succinate, hydroxypropyl methylcellulose phthalate, hydroxypropyl methylcellulose acetate succinate (hypromellose acetate succinate), polyvinyl acetate phthalate (PVAP), cellulose acetate trimellitate, and sodium alginate polymer.

For topical administration the alginate oligomer can be incorporated into creams, ointments, gels, salves, transdermal patches and the like. Further topical systems that are envisaged to be suitable are in situ drug delivery systems, for example gels where solid, semi-solid, amorphous or liquid crystalline gel matrices are formed in situ and which may comprise the alginate oligomer (which may be any alginate oligomer as herein defined). Such matrices can conveniently be designed to control the release of the alginate oligomer from the matrix, e.g. release can be delayed and/or sustained over a chosen period of time. Such systems may form gels only upon contact with biological tissues or fluids, e.g. mucosal surfaces. Typically the gels are bioadhesive and/or mucoadhesive. Delivery to any body site that can retain or be adapted to retain the pre-gel composition can be targeted by such a delivery technique. Such systems are described in WO 2005/023176), which is explicitly incorporated by reference herein in its entirety.

The relative content of the alginate oligomer in the compositions of the invention can vary depending on the dosage required and the dosage regime being followed but will be sufficient to achieve the requisite local concentration at the mucosal surface of the target treatment area, taking account of variables such as the physical size of the subject to be treated, the nature of the subject's particular ailments, and the location and identity of the target treatment area. The skilled man would know that the amounts of alginate can be reduced if a multiple dosing regime is followed or increased to minimise the number of administrations or applications.

A representative topical formulation, e.g. a cream, ointment or salve, which may be used to administer an alginate oligomer of the invention to the cervix or other parts of the lower female reproductive system might contain 1 to 25%, 1 to 20%, 1 to 15%, 1 to 10%, 1 to 9%, 1 to 8%, 1 to 7%, 1 to 6%, 5 to 25%, 5 to 20%, 5 to 15%, 5 to 10%, 5 to 9%, 5 to 8%, 5 to 7%, 5 to 6%, 8 to 25%, 8 to 20%, 8 to 15%, 8 to 10%, 9 to 25%, 9 to 20%, or 9 to 15% w/v of the oligomer, the remainder being comprised of pharmaceutically acceptable excipients, and/or other active agents if being used. Delivery devices designed for the application of topical formulations to the female reproductive system are known and may be employed to deliver the above mentioned formulations if convenient.

For administration to the nose or paranasal sinuses a sterile aqueous and/or oil-based liquid formulation (e.g. an emulsion) may be used; administered for instance by a nasal spray device, e.g. propellant-free or propellant-assisted. A representative formulation may contain 1 to 25%, 1 to 20%, e.g. 1 to 15%, 1 to 10%, 1 to 9%, 1 to 8%, 1 to 7% or 1 to 6%, 5 to 25%, 5 to 20%, 5 to 15%, 5 to 10%, 5 to 9%, 5 to 8%, 5 to 7%, 5 to 6%, 8 to 25%, 8 to 20%, 8 to 15%, 8 to 10%, 9 to 25%, 9 to 20%, or 9 to 15% w/v or w/w of the oligomer, the remainder being comprised of pharmaceutically acceptable excipients, e.g. water, and/or other active agents if being used.

In other embodiments a slow, delayed or sustained release formulations may be used for delivery, e.g. to the nose or paranasal sinuses. A representative formulation may be a powder containing the alginate oligomer or a suspension of said powder, said powder containing up to 90%, e.g. up to 85%, 80%, 75% or 70%, e.g. 50 to 90%, 55 to 90%, 60 to 90%, 65 to 90%, 70 to 90%, 75 to 90%, 80 to 90%, 85 to 90%, 50 to 85%, 55 to 85%, 60 to 85%, 65 to 85%, 70 to 85%, 75 to 85%, 80 to 85%, 50 to 80%, 55 to 80%, 60 to 80%, 65 to 80%, 70 to 80%, 75 to 80%, 50 to 70%, 55 to 70%, 60 to 70%, or 65 to 70%. The powder may comprise a coating that controls release of the alginate oligomer w/v or w/w of the oligomer, the remainder being comprised of pharmaceutically acceptable excipients and/or other active agents if being used.

A representative inhalable solution to be used to administer an alginate oligomer of the invention to the upper respiratory tract typically will be sterile and may contain 6 to 25%, e.g. 6 to 20%, 6 to 15%, 6 to 10%, 8 to 25%, 8 to 20%, 8 to 15%, 9 to 25%, 9 to 20%, 9 to 15%, 10 to 15%, 10 to 20%, 10 to 25%, 15 to 20% or 15 to 25% w/v of the oligomer, the remainder being comprised of pharmaceutically acceptable excipients, e.g. water, and/or other active agents if being used.

A representative inhalable powder to be used to administer an alginate oligomer of the invention to the lower respiratory tract may contain up to 90%, e.g. up to 85%, 80%, 75% or 70%, e.g. 50 to 90%, 55 to 90%, 60 to 90%, 65 to 90%, 70 to 90%, 75 to 90%, 80 to 90%, 85 to 90%, 50 to 85%, 55 to 85%, 60 to 85%, 65 to 85%, 70 to 85%, 75 to 85%, 80 to 85%, 50 to 80%, 55 to 80%, 60 to 80%, 65 to 80%, 70 to 80%, 75 to 80%, 50 to 70%, 55 to 70%, 60 to 70%, or 65 to 70% w/v or w/w of the oligomer, the remainder being comprised of pharmaceutically acceptable excipients and/or other active agents if being used.

A representative tablet to be used to administer an alginate oligomer of the invention to the lower GI tract may contain up to 99%, up to 95%, 90%, 85% or 80%, e.g. 50 to 95%, 55 to 95%, 60 to 95%, 65 to 95%, 70 to 95%, 75 to 95%, 80 to 95%, 85 to 95%, 90 to 95%, 50 to 90%, 50 to 90%, 55 to 90%, 60 to 90%, 65 to 90%, 70 to 90%, 75 to 90%, 80 to 90%, 85 to 90%, 50 to 90%, 55 to 85%, 60 to 80% or, 65 to 75% w/v or w/w of the oligomer, the remainder being comprised of pharmaceutically acceptable excipients and/or other active agents if being used.

An enteric coated tablet may also be effective in administering an alginate oligomer of the invention to the lower GI tract. A representative enteric coated tablet may contain up to 95%, e.g. up to 90%, 85% or 80%, e.g. 55 to 90%, 60 to 90%, 65 to 90%, 70 to 90%, 75 to 90%, 80 to 90%, 85 to 90%, 55 to 85%, 60 to 85%, 65 to 85%, 70 to 85%, 75 to 85%, 80 to 85%, 50 to 80%, 55 to 80%, 60 to 80%, 65 to 80%, 70 to 80%, or 75 to 80% w/v or w/w of the oligomer, the remainder being comprised of pharmaceutically acceptable excipients, including the enteric coating (e.g. polymers including fatty acids, waxes, shellac, plastics, and plant fibres) and/or other active agents if being used.

A pessary may be used to administer an alginate oligomer of the invention to the lower parts of the female reproductive tract. A representative formulation may contain 1 to 25%, 1 to 20%, e.g. 1 to 15%, 1 to 10%, 1 to 9%, 1 to 8%, 1 to 7%, 1 to 6%, 5 to 25%, 5 to 20%, 5 to 15%, 5 to 10%, 5 to 9%, 5 to 8%, 5 to 7%, 5 to 6%, 8 to 25%, 8 to 20%, 8 to 15%, 8 to 10%, 9 to 25%, 9 to 20%, or 9 to 15% w/v or w/w of the oligomer, the remainder being comprised of pharmaceutically acceptable excipients, including solid excipients (e.g. paraffin and the like), and/or other active agents if being used.

A representative aqueous solution for direct delivery to a mucosal surface in the liver, the pancreas or the reproductive system will be sterile and may contain 6 to 25%, e.g. 6 to 20%, 6 to 15%, 6 to 10%, 8 to 25%, 8 to 20%, 8 to 15%, 9 to 25%, 9 to 20%, 9 to 15%, 10 to 15%, 10 to 20%, 10 to 25%, 15 to 20%, or 15 to 25% w/v of the oligomer, the remainder being comprised of water and pharmaceutically acceptable excipients and/or other active agents if being used.

The invention will be further described with reference to the following non-limiting Examples in which:

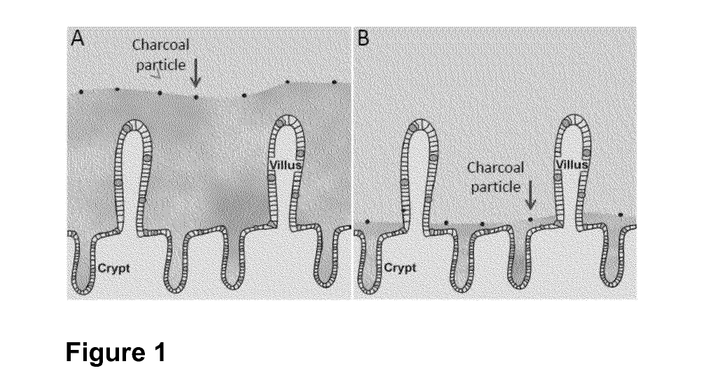

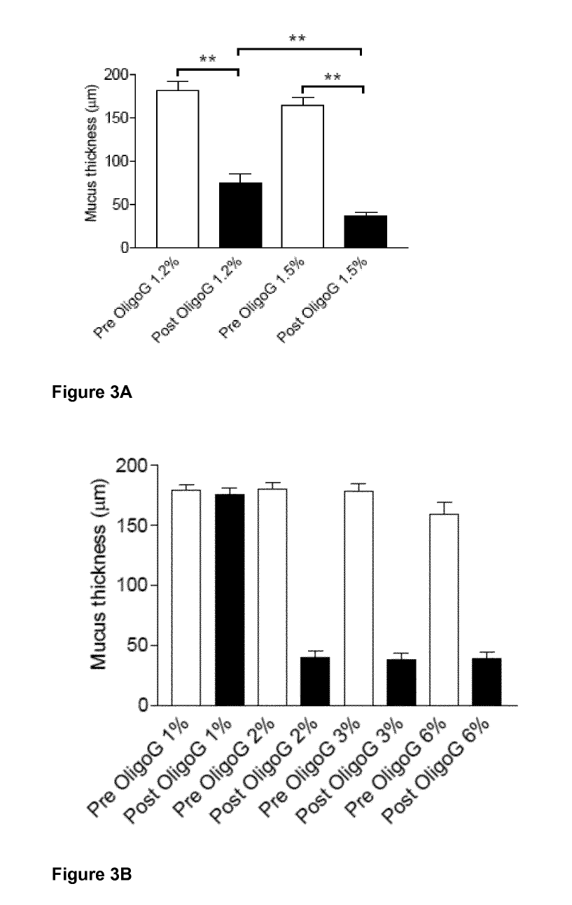

FIG. 1 shows a diagrammatic representation of the technique employed in the Examples to measure ileum mucus thickness. (A) Initial mucus thickness was measured from the charcoal particles on the mucus to the villi tips (“Pre” in FIGS. 3, 5, 6 and 7). After removal, mucus thickness was measured again from the charcoal particles to the villi tips (“Post” in FIGS. 3, 5, 6 and 7). (B) In order to establish total mucus thickness, all mucus was removed, new charcoal and Krebs-mannitol were added to the chamber and villus height was measured from charcoal to villus tip.

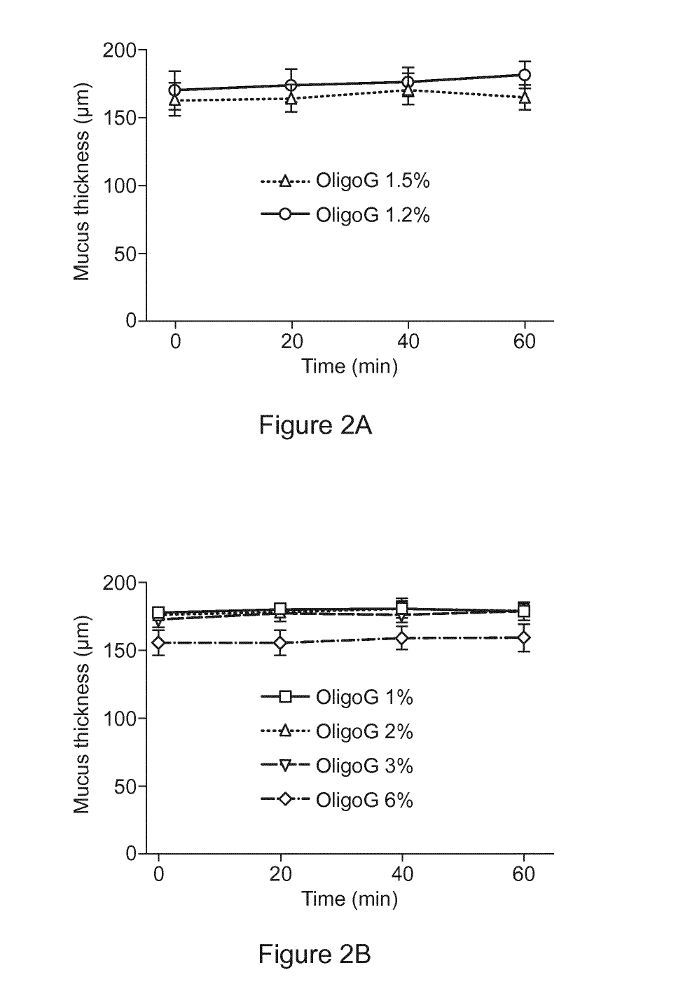

FIG. 2 shows that mucus thickness was not affected by incubation with 1%, 1.2%, 1.5%, 2%, 3% or 6% apical OligoG. Krebs-mannitol containing 1.2% (circles) or 1.5% (triangle) OligoG (2A) or 1% (squares), 2% (triangles), 3% (inverted triangles) or 6% (diamond) OligoG (2B) was incubated for 1 hour on already formed mucus and mucus thickness measured every 20 minutes for one hour. 1%, n=3; 1.2% and 1.5%, n=5; 2% and 3%, n=6; and 6%, n=3.

FIG. 3 shows that mucus attachment decreases after incubation for one hour with increasing concentrations of OligoG. Ileal explants from CftrΔF508 mutant mice were covered by Krebs-mannitol containing 1.2% or 1.5% OligoG (3A) or 1%, 2%, 3% or 6% OligoG (3B) and the thickness of mucus on the mucosal side of the explants was measured. After 1 hr incubation standard mucus removal was performed and thickness measured. Mucus thickness before (Pre; white bars) and after (Post; black bars) standardized removal of mucus is illustrated. 1%, n=3; 1.2% and 1.5%, n=5 (P=0.008); 2% and 3%, n=6; and 6%, n=3.

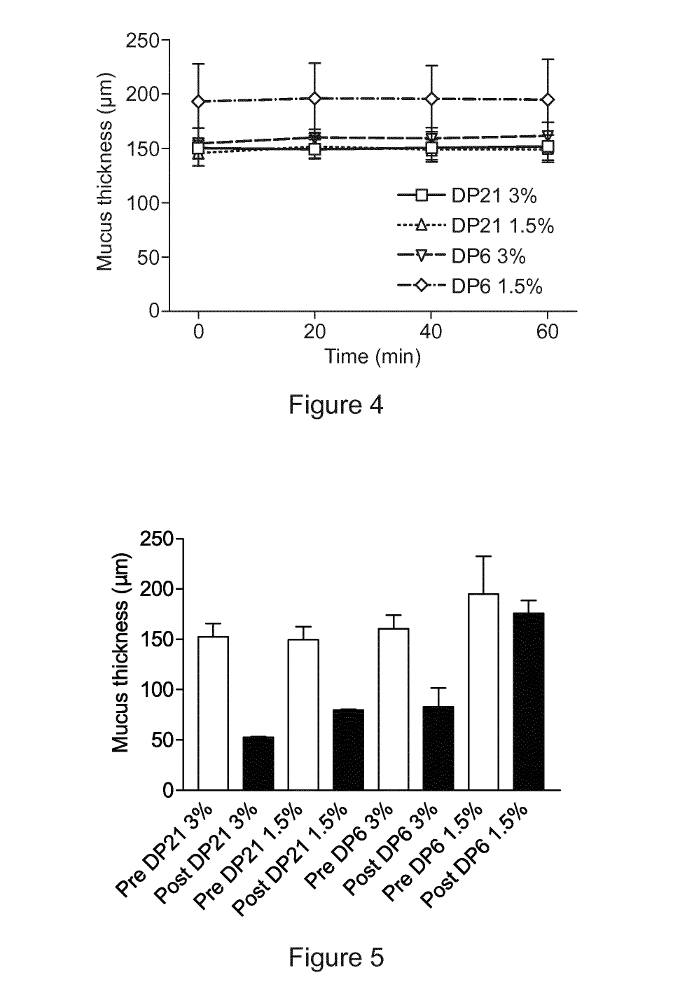

FIG. 4 shows that mucus thickness was not affected by incubation with 1.5% or 3% apical alginate oligomer (6mer, 88% G or 21 mer, 88% G). Krebs-mannitol containing 1.5% 6mer (diamonds), 3% 6mer (inverted triangles), 1.5% 21mer (triangles) and 3% 21mer (squares) were incubated for 1 hour on already formed mucus and mucus thickness measured every 20 minutes for one hour.

FIG. 5 shows that mucus attachment decreases after incubation for one hour with 1.5% or 3% apical alginate oligomer (6mer, 88% G (DP6) or 21 mer 88% G (DP21)). Ileal explants from CftrΔF508 mutant mice were covered by Krebs-mannitol containing 1.5% or 3% oligomer and the thickness of mucus on the mucosal side of the explants was measured. After 1 hr incubation standard mucus removal was performed and thickness measured. Mucus thickness before (Pre; white bars) and after (Post; black bars) standardized removal of mucus is illustrated.

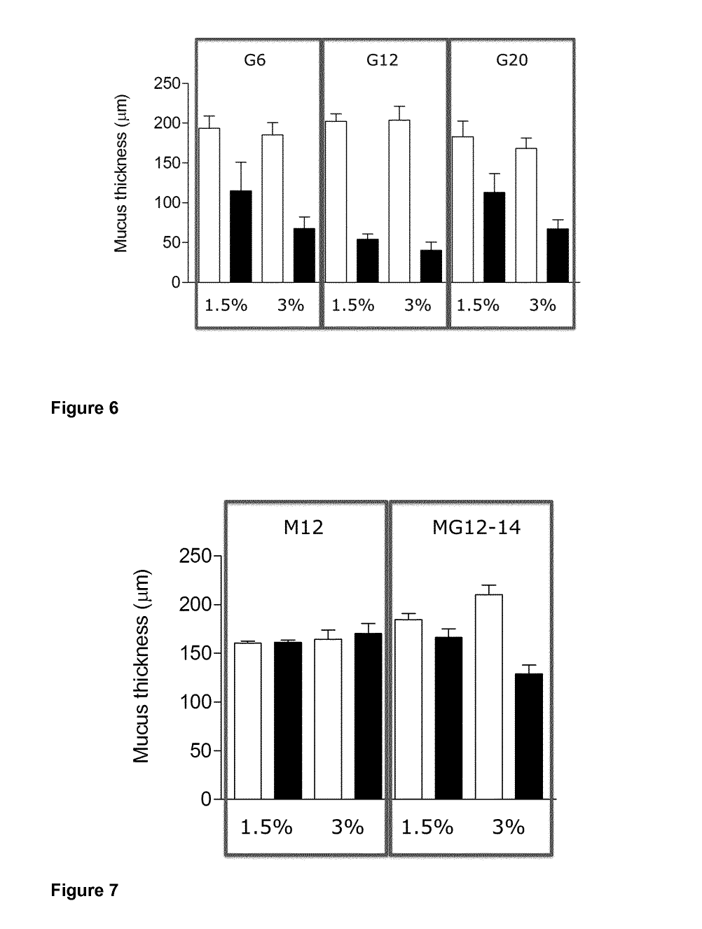

FIG. 6 shows that mucus attachment decreases after incubation for one hour with 1.5% or 3% apical alginate oligomer (6mer (G6), 12mer (G12), or 20mer (G20); all containing at least 85% G residues). Ileal explants from CftrΔF508 mutant mice were covered by Krebs-mannitol containing 1.5% or 3% oligomer and the thickness of mucus on the mucosal side of the explants was measured. After 1 hr incubation standard mucus removal was performed and thickness measured, n=4. Mucus thickness before (Pre; white bars) and after (Post; black bars) standardized removal of mucus is illustrated.

FIG. 7 shows that mucus attachment does not decrease, or decreases slightly, after incubation for one hour with 1.5% or 3% apical alginate oligomer (12mer, 100% M (M12); 12 to 14mers containing approximately equal amounts of M and G in alternating sequence (MG12-14)). Ileal explants from CftrΔF508 mutant mice were covered by Krebs-mannitol containing 1.5% or 3% oligomer and the thickness of mucus on the mucosal side of the explants was measured. After 1 hr incubation standard mucus removal was performed and thickness measured, n=4. Mucus thickness before (Pre; white bars) and after (Post; black bars) standardized removal of mucus is illustrated.

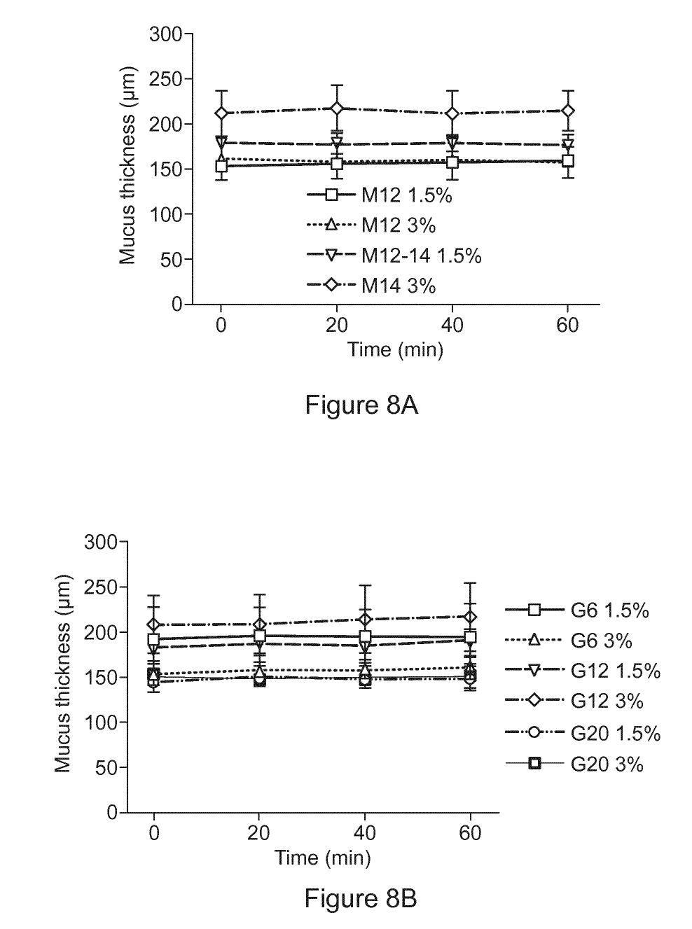

FIG. 8 shows that mucus thickness was not affected by incubation with 1.5% or 3% apical alginate oligomer: 8A—12mer, 100% M (M12—squares and triangles, respectively); 12 to 14mers containing approximately equal amounts of M and G in alternating sequence (MG12-14—inverted triangles and diamonds, respectively); 8B—timer containing at least 85% G residues (G6—solid squares and triangles, respectively); 12mer containing at least 85% G residues (G12—inverted triangles and diamonds, respectively); or 20mer containing at least 85% G residues (G20—circles and empty squares, respectively)). Krebs-mannitol containing alginate oligomers were incubated for 1 hour on already formed mucus and mucus thickness measured every 20 minutes for one hour.

### EXAMPLES

### Example 1

### Materials and Standard Methods

Animals

Homozygous CftrΔF508 mice on C57BL/6 background (backcrossed for 13 generations) were kept under specific pathogen free conditions in individually ventilated cages under controlled temperature (21-22° C.), humidity and 12 h light/dark cycle, maintained on special chow and water with PEG and salts to avoid distal ileal obstruction ad libitum, and given regular water 4-7 days before the experiments. Animals were generated by heterozygous breeding, biopsied at weaning and genotyped with a PCR based method on genomic DNA prepared from the biopsies. The PCR product was cleaved with a restriction enzyme and the ensuing pattern on the agarose gel used to classify the animals. Ethical approval for the animal experiments was granted by the Ethics Committee for Animal experiments in Gothenburg.

Explant Tissue

Mice were euthanized by isoflurane and cervical dislocation. The distal ileum was dissected and flushed with ice-cold 95% O₂/5% CO₂ Krebs solution (116 mM NaCl, 1.3 mM CaCl₂, 3.6 mM KCl, 1.4 mM KH₂PO₄, 23 mM NaHCO₃, and 1.2 mM MgSO₄), pH 7.4, and kept on ice during transportation (30 min). The tissue was opened along the mesenteric border, the longitudinal smooth muscle was removed, and the tissue was divided into two pieces and mounted in a horizontal Ussing-type perfusion chamber (Gustafsson, J. K., et al supra) with a circular opening of 4.9 mm². The chamber was mounted in a heating block connected to a temperature controller (Harvard Apparatus, Holliston, Mass.), allowing the experiments to be performed at 37° C. The apical solution was kept unstirred to avoid disturbances to the mucus gel, whereas the serosal chamber was constantly perfused at a rate of 5 ml/h. Trans-epithelial potential difference (PD) was measured during the whole experiment using Calomel electrodes (Ref201; Radiometer, Copenhagen, Denmark) connected to the tissue bath via agar bridges (4% agar, 0.9% NaCl). The serosal chamber was constantly perfused with 95% O₂/5% Krebs solution containing 10 mM glucose, 5.1 mM Na-glutamate, and 5.7 mM Na-pyruvate (Krebs-glucose). The apical chamber was filled with 150 μl likewise 95% O₂/5% CO₂-bubbled Krebs solution where glucose was substituted with 10 mM D-mannitol (Krebs-mannitol). After bubbling with 95% O₂/5% CO₂, the pH of these solutions was 7.4. The two adjacent parts of the tissue were analysed in parallel.

Mucus Thickness Measurements

The mucus surface was visualized by activated charcoal particles (Fluka, Sigma-Aldrich, Stockholm, Sweden). Mucus thickness was assessed by measuring the distance between the charcoal particle and the villus tip (D) using a micropipette (OD: 1.2 mm, ID: 0.6 mm) pulled to a tip diameter of 5-10 μm. The micropipette was mounted in a micromanipulator (in house) connected to a digimatic indicator (Mitotoyo, Tokyo, Japan). The tissue was viewed through a stereomicroscope at 40× magnification (Leica MZ125, Leica, Wetzlar, Germany). The level of the epithelial surface was determined as the point where the tip of the micropipette and the epithelial surface was in the same focal plane. The micropipette was kept at a constant angle of 40° and the vertical thickness of the mucus was obtained by multiplying the distance (D) with cos 40. Five measurements were made for each time point, and the mean thickness was calculated and used as a single value.

Mucus Adhesion Measurements

The adhesiveness of the mucus layer was assessed by comparing the total mucus thickness to the mucus thickness remaining after aspiration. The aspiration procedure was performed in a standardized way by using a small plastic Pasteur pipette (PP-101, outer tip diameter 0.9 mm, inner tip diameter 0.7 mm, max volume 800 μl; Cell Projects, Harrietsham, UK). The tip of the compressed pipette was placed on the edge of the chamber opening and slowly opened over three seconds to aspirate the apical chamber solution and the non-adherent mucus. The size of the pipette allows for removal of the whole apical solution in one step. The remaining mucus thickness was measured after refilling the apical chamber with 150 μl Krebs-mannitol and the addition of new charcoal particles. After total removal of the mucus layer, the villus height was assessed by measuring the distance between the villus tip and the surface epithelium in between the villi. Total mucus thickness is presented as the sum of these two measurements (FIG. 1).

Statistics Results are presented as mean±SEM. Number of animals in each group is denoted n. Differences were tested using Mann-Whitney U test.

### Example 2

### Measurement of Mucus Thickness Upon Incubation with OligoG

After mounting explants from CftrΔF508 mutant mice in the Ussing-type chamber, Krebs-mannitol buffer (pH 7.4) containing 1.2% and 1.5% OligoG (13mer, 93% G) was added to the apical chamber. Mucus thickness on the ileal explants was measured every 20 minutes for 1 hour. FIG. 2A illustrates the absence of change in mucus thickness over this time. Both groups contain 5 animals. Experiment was repeated with OligoG concentrations of 1%, 2%, 3% or 6%. FIG. 2B illustrates the absence of change in mucus thickness over the treatment period. In the 1% group there were 3 animals, the 2% and 3% groups consist of 6 animals in each and the 6% group is made up of 3 animals.

Incubation with concentrations of OligoG at 1%, 1.2%, 1.5%, 2%, 3%, or 6% did not cause an increase in mucus thickness of already formed mucus over one hour at 37° C.

### Example 3

### Measurement of Mucus Thickness Upon Standardised Removal of Mucus Following Incubation with OligoG

Ileal explants from CftrΔF508 mutant mice were incubated for 1 hour in increasing concentrations of OligoG, during which mucus thickness was measured every 20 minutes. At one hour standardised removal of mucus was performed to assess mucus attachment to the epithelium. FIGS. 3A and 3B together show mucus thickness before (Pre) and after (Post) aspiration (removal) in explants exposed to 1%, 1.2%, 1.5%, 2%, 3%, or 6% OligoG. Mucus is still attached after incubation with 1% OligoG. After a one-hour incubation with 1.2% OligoG it was possible to almost completely remove the mucus and after a one-hour incubation with 1.5%, 2%, 3% or 6% OligoG it was possible to completely remove the mucus. This indicates that the mucus has undergone a transformation into a more normal (detached) phenotype. Macroscopic examination of the tissue under the stereo microscope reveals no obvious damage after incubation with OligoG.

### Example 4

### Measurement of Mucus Thickness Upon Incubation with G-Rich Alginate Oligomers of Varying Size

After mounting explants from CftrΔF508 mutant mice in the Ussing-type chamber, Krebs-mannitol buffer (pH 7.4) containing either 1.5% or 3% alginate oligomer (6mer, 88% G or 21 mer, 88% G) was added to the apical chamber. Mucus thickness on the ileal explants was measured every 20 minutes for 1 hour. FIG. 4 illustrates that incubation with either 1.5% or 3% concentrations of either alginate oligomer did not cause an increase in mucus thickness of already formed mucus over one hour at 37° C.

### Example 5

### Measurement of Mucus Thickness Upon Standardised Removal of Mucus Following Incubation with G-Rich Alginate Oligomers of Varying Size

Ileal explants from CftrΔF508 mutant mice were incubated for 1 hour in 1.5% or 3% alginate oligomer (6mer, 88% G or 21 mer, 88% G), during which mucus thickness was measured every 20 minutes. At one hour standardised removal of mucus was performed to assess mucus attachment to the epithelium. FIG. 5 shows mucus thickness before (Pre) and after (Post) aspiration (removal) in explants exposed to 1.5% or 3% of each alginate oligomer. After a one-hour incubation with 3% of each oligomer it was possible to remove at least 50% of the mucus. A 1.5% concentration of the 21 mer was slightly less effective than a 3% concentration of the 21 mer, but more effective than a 3% concentration of the 6mer. A 1.5% concentration of the 6mer was the least effective of the treatments tested, but nevertheless partial detachment is seen.

### Example 6

### Measurement of Mucus Thickness Upon Standardised Removal of Mucus Following Incubation with G-Rich Alginate Oligomers of Varying Size

Ileal explants from CftrΔF508 mutant mice (as used and handled in preceding Examples) were incubated for 1 hour in 1.5% or 3% alginate oligomer (6mer, 12mer or 20mer all containing at least 85% G residues), during which mucus thickness was measured every 20 minutes. At one hour standardised removal of mucus was performed to assess mucus attachment to the epithelium. FIG. 6 shows mucus thickness before (Pre) and after (Post) aspiration (removal) in explants exposed to 1.5% or 3% of each alginate oligomer.

All sizes of G-rich oligomers tested caused mucus detachment at 1.5% and 3% concentrations. In all instances a 3% concentration was more effectivce than the 1.5% concentration. The 6mer and the 20mer gave similar results overall. The 12mer was noticeably more effective than the other sizes at both 1.5% and 3% concentrations. Indeed, treatment with the 12mer made it possible to completely aspirate the mucus at both 1.5% and 3% concentrations, indicating a transformation of the mucus into a more normal phenotype. Clearly, the 6mer and 20mer oligomers can achieve this end to varying degrees depending on the concentration. At a concentration of 3% detachment induced by the 6mer or the 20mer is almost as complete as with a 1.5% concentration of 12mer, i.e. a phenotype that is considered to be normal.

### Example 7

### Measurement of Mucus Thickness Upon Standardised Removal of Mucus Following Incubation with an M-Rich Alginate Oligomer and an Alginate Oligomer of Alternating M/G Residues

Ileal explants from CftrΔF508 mutant mice (as used and handled in preceding Examples) were incubated for 1 hour in 1.5% or 3% alginate oligomer (12mer, 100% M; 12 to 14mers containing approximately equal amounts of M and G in alternating sequence), during which mucus thickness was measured every 20 minutes. At one hour standardised removal of mucus was performed to assess mucus attachment to the epithelium. FIG. 7 shows mucus thickness before (Pre) and after (Post) aspiration (removal) in explants exposed to 1.5% or 3% of each alginate oligomer.

In this assay, mucus was as attached as in the CF mouse after incubation with 1.5% or 3% concentrations of the high M 12mer. 1.5% of the MG 12 to 14mers also showed little effect on mucus attachment in this assay. When the concentration of the MG 12 to 14mers was increased to 3%, the mucus was a little more easily aspirated than in the CF ileum, but still more attached than in the WT ileum. Greater effects may be seen at higher concentrations, e.g. towards 6%.

### Example 8

### Measurement of Mucus Thickness Upon Incubation with Alginate Oligomers of Varying Sizes and M and G Contents

After mounting explants from CftrΔF508 mutant mice in the Ussing-type chamber (as previously described), Krebs-mannitol buffer (pH 7.4) containing either 1.5% or 3% alginate oligomer (timer containing at least 85% G residues; 12mer containing at least 85% G residues; 20mer containing at least 85% G residues; 12mer, 100% M; 12 to 14mer containing approximately equal amounts of M and G in alternating sequence) was added to the apical chamber. Mucus thickness on the ileal explants was measured every 20 minutes for 1 hour. FIGS. 8A and 8B illustrate that incubation with either 1.5% or 3% concentrations of the tested alginate oligomers did not cause an increase in mucus thickness of already formed mucus over one hour at 37° C.

1. A method for the treatment of a condition in a human patient arising from or associated with a defective cystic fibrosis transmembrane conductance regulator (CFTR) ion channel and/or abnormal mucus which is attached to underlying epithelium, said method comprising administering an alginate oligomer, wherein at least 30% of the monomer residues of the alginate oligomer are G residues, to the patient in an amount sufficient to achieve a local concentration of the alginate oligomer of 1 to 6% w/v at at least part of a mucosal surface with a defective CFTR ion channel and/or said abnormal mucus in the patient, thereby to result in at least partial detachment of mucus from said mucosal surface.

2. (canceled)

3. The method of claim 1, wherein said condition is a respiratory disorder or a complication thereof.

4. The method of claim 3, wherein the respiratory disorder is an obstructive respiratory disorder, or wherein the respiratory disorder is characterized by a chronic inflammatory state, airway remodelling and exacerbations due to respiratory tract infections.

5. The method of claim 1, wherein said condition is cystic fibrosis (CF), non-compound CFTR gene mutation heterozygosity, abnormal mucus clearance in the respiratory tract and/or breathing difficulties resulting from chronic particulate inhalation, COPD, chronic bronchitis, emphysema, bronchiectasis, asthma or chronic sinusitis, or a complication thereof.

6. The method of claim 1, wherein said condition is CF or a complication thereof.

7. The method of claim 1, wherein said mucosal surface is selected from a mucosal surface in the:
(i) respiratory tract, preferably trachea, bronchi or bronchioles,
(ii) paranasal sinus,
(iii) GI tract, preferably the mouth, pharynx, oesophagus, duodenum, jejunum or ileum,
(iv) pancreas, preferably the pancreatic ducts,
(v) liver, preferably the bile ducts, or
(vi) reproductive system, preferably the cervix, uterus or fallopian tubes or the epididymis or vas deferens.

8. The method of claim 1, wherein said treatment comprises the treatment or prevention of a complication of said condition, wherein said complication is selected from a complication of
(i) the respiratory tract and/or cardiovascular system;
(ii) a paranasal sinus;
(iii) the GI tract,
(iv) the pancreas;
(v) the liver; or
(vi) fertility.

9. The method of claim 8, wherein said complication is or involves an infection of the respiratory tract or results from stagnant mucus in the GI tract.

10. The method of claim 1, wherein the local concentration of the alginate oligomer is 1.2 to 6% w/v, 1.5 to 6% w/v or 2 to 6% w/v.

11. The method of claim 1, wherein said alginate oligomer has an average molecular weight of less than 35,000 Daltons, preferably less than 30,000, 25,000 or 20,000 Daltons.

12. The method of claim 1, wherein the alginate oligomer has a degree of polymerisation (DP), or a number average degree of polymerisation (DPn) of 4 to 100, 4 to 75, 4 to 50, 4 to 35, 4 to 30, 4 to 25, 4 to 22, 4 to 20, 4 to 18, 4 to 16 or 4 to 14.

13. The method of claim 1, wherein the alginate oligomer has a degree of polymerisation (DP), or a number average degree of polymerisation (DPn) of
(i) 6 to 50, 6 to 35, 6 to 30, 6 to 25, 6 to 22, 6 to 20, 6 to 18, 6 to 16 or 6 to 14, or
(ii) 8 to 50, 8 to 35, 8 to 30, 8 to 25, 10 to 25, 10 to 22, 10 to 20, 10 to 18, or 10 to 15.

14. The method of claim 1, wherein the alginate oligomer has at least 70% G residues.

15. The method of claim 14, wherein the alginate oligomer has at least 80%, or at least 85%, or at least 90%, or at least 95% G residues.

16. The method of claim 1, wherein at least 80% of the G residues are arranged in G-blocks.

17. The method of claim 1, wherein said alginate oligomer is used in combination with a further therapeutically active agent for the treatment of condition arising from or associated with a defective cystic fibrosis transmembrane conductance regulator (CFTR) ion channel and/or abnormal mucus which is attached to underlying epithelium, said further therapeutically active agent being selected from an antibiotic, an antifungal, an antiviral, an immunostimulatory agent, a corticosteroid, a non-steroidal antiinflammatory drug (NSAID), a bronchodilator, a digestive enzyme supplement, an oral antidiabetic drug, an injectable antidiabetic drug and a mucolytic agent.

18. A product containing an alginate oligomer, wherein at least 30% of the monomer residues of the alginate oligomer are G residues and a further comprising therapeutically active agent for the treatment of condition arising from or associated with a defective cystic fibrosis transmembrane conductance regulator (CFTR) ion channel and/or abnormal mucus which is attached to underlying epithelium, said further therapeutically active agent being selected from an antibiotic, an antifungal, an antiviral, an immunostimulatory agent, a corticosteroid, a non-steroidal antiinflammatory drug (NSAID), a bronchodilator, a digestive enzyme supplement, an oral antidiabetic drug, an injectable antidiabetic drug and a mucolytic agent as a combined preparation for separate, simultaneous or sequential use in the treatment of a condition in a human patient arising from or associated with a defective CFTR ion channel and/or abnormal mucus which is attached to underlying epithelium.
